(19) United States
(12) Patent Application Publication
(10) Pub. No.: US 2025/0361558 A1
(43) Pub. Date: Nov. 27, 2025

(54) THIRD DNA BASE PAIR SITE-SPECIFIC DNA DETECTION

(71) Applicant: ILLUMINA, INC., San Diego, CA (US)

(72) Inventors: Xiaolin WU, Cambridge (GB); Xiaohai LIU, Cambridge (GB); Colin Brown, San Diego, CA (US); Sarah Shultzaberger, San Diego, CA (US); Eric Brustad, San Diego, CA (US)

(21) Appl. No.: 18/874,535

(22) PCT Filed: Jul. 28, 2023
(86) PCT No.: PCT/US2023/028999

§ 371 Date: Dec. 12, 2024

Related U.S. Application Data
(60) Provisional application No. 63/399,339, filed on Aug. 19, 2022.

(51) Int. Cl. C12Q 1/6876; C12Q 1/6806

(52) U.S. Cl. CPC C12Q 1/6876; C12Q 1/6806; C12Q 2600/154

(57) ABSTRACT

Embodiments of the present disclosure relate to six-nucleobase libraries having a third Watson-Crick base pair. Also provided herein are methods to prepare such six-nucleobase libraries, and their use for sequencing and modified nucleobase detection applications.

### BACKGROUND

### Field

The present disclosure generally relates to the site-specific detection of modified nucleobases including 5-methylcytosine in polynucleotides. More particularly, the present disclosure relates to six-nucleobase nucleotides that contain a novel third base pair and their use in six-nucleobase polynucleotide sequencing and detection methods. Methods of preparing the six-nucleobase nucleotides and six-nucleobase nucleosides, six-nucleobase polynucleotides, or six-nucleobase oligonucleotides are also disclosed.

### Description of the Related Art

Methylation of cytosine nucleobases at the C-5 position of the pyrimidine ring is an important epigenetic marker in genomic DNA and is proposed to have diverse roles in regulation of gene expression, parental imprinting, and molecular etiology of human diseases such as cancer or diabetes.

A traditional detection method of 5-methylcytosine nucleobases is whole-genome bisulfite sequencing (WGBS), which detects methylated nucleobases by the absence of conversion, and can be considered an “inverse detection” assay. When bisulfite-treated DNA is sequenced, unmodified cytosine nucleobases can be identified as cytosine-to-thymine mutations, whereas 5-methylcytosine nucleobases are read as cytosine. This in effect creates a “three-base genome”, masking cytosine-to-thymine and thymine-to-cytosine single nucleotide polymorphisms (SNPs) that results in overestimation of 5-methylcytosine abundance. Side reactions during the WGBS process can result in cleavage of the DNA backbone, leading to dropout of regions of the genome with a high proportion of nonmethylated cytosine nucleobases that results in GC bias. These issues prevent whole-genome sequencing for SNP detection of WGBS samples, and require the preparation of a parallel whole-genome sequencing (WGS) library. In cases when a minimal amount of analyte prevents the creation of the parallel library simultaneous detection of 5-methylcytosine and SNPs may not be possible. Furthermore, WGBS and other next-generation sequencing-based (NGS) methods for detection of 5-methylcytosine rely on cytosine-to-uracil conversion to mark modified positions, which masks cytosine-to-thymine SNPs and precludes simultaneous methylation detection and variant calling.

### SUMMARY

Some embodiments provided herein relate to methods of detecting a modified nucleobase in a target polynucleotide strand. In some embodiments, the methods include detecting 5-methylcytosine in a target polynucleotide strand. In some embodiments, the methods include providing a target polynucleotide strand comprising the modified nucleobase. In some embodiments, the modified nucleobase is 5-methylcytosine. In some embodiments, the methods include forming a copy polynucleotide strand comprising a paired nucleobase. In some embodiments, the methods include removing the modified nucleobase. In some embodiments, the methods include converting the paired nucleobase into an orthogonal nucleobase. In some embodiments, the methods include incorporating a signal nucleotide into a signal polynucleotide strand. The signal nucleotide comprises a signal nucleobase and a detectable label.

In some embodiments, the signal nucleobase comprises the structure:

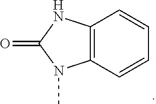

In some embodiments, signal nucleobase does not achieve Watson-Crick base pairing with a natural nucleobase.

Tn some embodiments, the orthogonal nucleobase has the structure selected from:

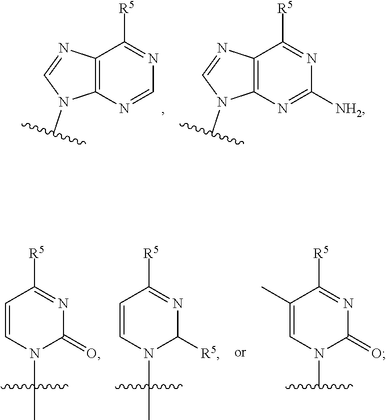

and
wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl. In some embodiments, the orthogonal nucleobase is O-benzylguanine. In some embodiments, the orthogonal nucleobase does not achieve Watson-Crick base pairing with the natural nucleobase.

In some embodiments, the modified nucleobase is selected from the group consisting of a modified adenine, a modified cytosine, a modified guanine, and a modified thymine, and a modified uracil. In some embodiments, the paired nucleobase is selected from the group consisting of adenine, cytosine, guanine, thymine, and uracil.

In some embodiments, the removing is accomplished by a glycosylase selected from the group consisting of ROS1 DNA glycosylase, DME DNA glycosylase, DML2 DNA glycosylase, and DML3 DNA glycosylase.

In some embodiments, converting the paired nucleobase is accomplished with chemical reagents. In some embodiments, the chemical reagents comprising a diazo compound having the structure N₂CWZ. In some embodiments, W is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, or an optionally substituted derivative of any of the foregoing. In some embodiments, Z is selected from C(O)NR¹R², C(O)OR¹, C(O)SR¹, C(S)OR¹, and C(S)SR¹. In some embodiments, R¹ and R² are independently selected from C₁-C₁₂ alkyl, C₂-C₁₂ alkenyl, C₂-C₁₂ alkynyl, C₁-C₁₂ alkoxy, C₁-C₁₂ heteroalkyl, cyano, halo, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, C₁-C₁₂ thioalkyl, C₁-C₁₂ sulfonyl, or an optionally substituted derivative of any of the foregoing. In some embodiments, R¹ and R² together optionally are 3-10 membered heterocyclyl or 5-10 membered heteroaryl. In some embodiments, the chemical reagents add a functional group to the paired nucleobase, the functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

In some embodiments, the copy polynucleotide strand is a sulfur-containing copy nucleotide strand and forming the sulfur-containing copy polynucleotide strand is accomplished with 6-thioguanine deoxynucleotide triphosphate. In some embodiments, the paired nucleobase is a sulfur-containing paired nucleobase and converting the sulfur-containing paired nucleobase is accomplished with chemical reagents, the chemical reagents comprising one or more oxidizing agents and a nucleophile having the formula R⁴B¹, wherein B¹ is NH₂, OH, or SH and R⁴ is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl. In some embodiments, the chemical reagents add a functional group to the sulfur-containing paired nucleobase, the functional group having the formula R⁴B², wherein B² is NH, O, or S and R⁴ is selected from C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl.

In some embodiments, incorporating the plurality of signal nucleobases into the signal polynucleotide strand is accomplished using a polymerase. In some embodiments, the polymerase comprises an A-family DNA polymerase, a B-family DNA polymerase, a Y-family DNA polymerase, or combinations of any of the foregoing. In some embodiments, the polymerase is selected from the group consisting of Dpo4, Therminator, DeepVent® (exo−), KOD, KlenTaq, and KTqM747K.

Some embodiments provided herein relate to methods of detecting a modified nucleobase in a target polynucleotide strand. In some embodiments, the methods include providing a target polynucleotide strand comprising the modified nucleobase. In some embodiments, the methods include converting the modified nucleobase into a linked signal nucleobase. In some embodiments, the methods include incorporating an orthogonal nucleotide into a copy polynucleotide strand. The orthogonal nucleotide includes a linked orthogonal nucleobase. In some embodiments, the methods include incorporating a signal nucleotide into a signal polynucleotide strand. The signal nucleotide includes the linked signal nucleobase and a detectable label. In some embodiments, the linked signal nucleobase has the structure:

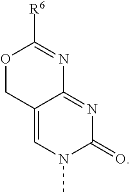

In some embodiments, R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing, In some embodiments, “ [symbol] ” the signal polynucleotide strand. In some embodiments, the liked orthogonal nucleobase has the structure:

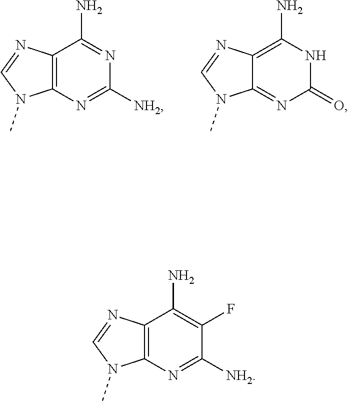

In some embodiments, “ [symbol] ” is a bond to the copy polynucleotide strand.

Some embodiments provided herein relate to methods of forming a six-nucleobase polynucleotide. In some embodiments, the six-nucleobase polynucleotide comprises a signal polynucleotide strand and a copy polynucleotide strand. In some embodiments, the signal polynucleotide strand comprises a plurality of signal nucleobases. In some embodiments, the copy polynucleotide strand comprises a plurality of orthogonal nucleobases. In some embodiments, a signal nucleobase comprises a structure selected from the group consisting of:

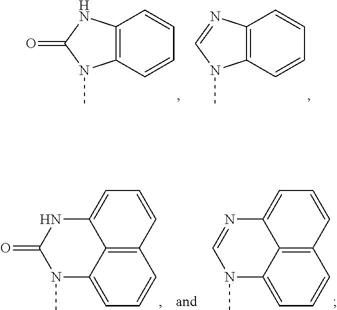

wherein “ [symbol] ” is a bond to the signal polynucleotide strand. In some embodiments, an orthogonal [symbol] nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl. In some embodiments, the orthogonal nucleobase achieves Watson-Crick base pairing with the signal nucleobase. In some embodiments, the methods include providing a target polynucleotide strand comprising the plurality of modified nucleobases. In some embodiments, the methods include forming the copy polynucleotide strand, the copy polynucleotide strand comprising the plurality of paired nucleobases. In some embodiments, the methods include removing the plurality of modified nucleobases to form a gapped polynucleotide strand. In some embodiments, the methods include converting the plurality of paired nucleobases into the plurality of orthogonal nucleobases. In some embodiments, the methods include incorporating the plurality of signal nucleobases into the signal polynucleotide strand.

Tn other embodiments, the signal polynucleotide strand comprises a plurality of linked signal nucleobases. In some embodiments, a linked signal nucleobase has the structure:

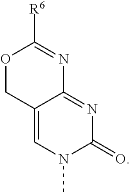

In some embodiments, R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, “ [symbol] ” is a bond to the signal polynucleotide strand. In some embodiments, the copy polynucleotide strand comprises a plurality of linked orthogonal nucleobases. In some embodiments, a linked orthogonal nucleobase has a structure selected from the group consisting of:

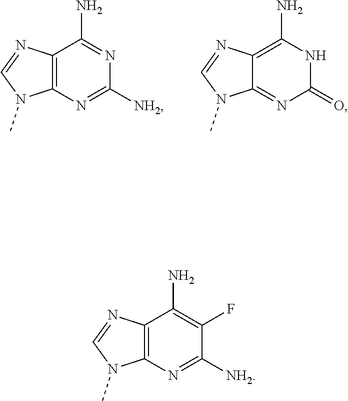

In some embodiments, “ [symbol] ” is a bond to the copy polynucleotide strand. In some embodiments, [symbol] the methods include providing a target polynucleotide strand comprising the plurality of modified nucleobases. The methods include converting the plurality of modified nucleobases into the plurality of linked signal nucleobases. The methods include incorporating a plurality of orthogonal nucleotides into the copy polynucleotide strand, wherein an orthogonal nucleotide comprises the linked orthogonal nucleobase. The methods include incorporating a plurality of signal nucleotides into the signal polynucleotide strand, wherein a signal nucleotide comprises the linked signal nucleobase and a detectable label.

Some embodiments provided herein relate to six-nucleobase polynucleotides. In some embodiments, the six-nucleobase polynucleotides comprise a signal polynucleotide strand and a copy polynucleotide strand. In some embodiments, the signal polynucleotide strand comprises a plurality of signal nucleobases. In some embodiments, the copy polynucleotide strand comprises a plurality of orthogonal nucleobases. In some embodiments, a signal nucleobase comprises a structure selected from the group consisting of:

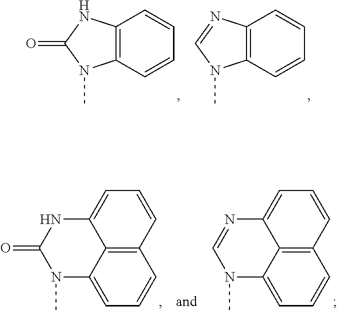

wherein “ [symbol] ” is a bond to the signal polynucleotide strand. In some embodiments, an orthogonal nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl. In some embodiments, the orthogonal nucleobase achieves Watson-Crick base pairing with the signal nucleobase.

In some embodiments, the signal nucleobase comprises the structure:

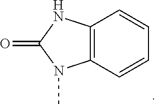

In some embodiments, the signal nucleobase does not achieve Watson-Crick base pairing with a natural nucleobase.

In some embodiments, the orthogonal nucleobase has the structure selected from:

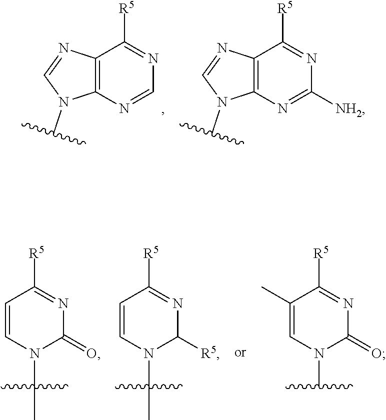

and
wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl. In some embodiments, the orthogonal nucleobase is O-benzylguanine. In some embodiments, the orthogonal nucleobase does not achieve Watson-Crick base pairing with a natural nucleobase.

In other embodiments, the signal polynucleotide strand comprises a plurality of linked signal nucleobases. In some embodiments, a linked signal nucleobase has the structure:

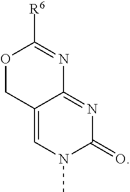

In some embodiments, R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, “ [symbol] ” is a bond to the signal polynucleotide strand. In other embodiments, the copy polynucleotide strand comprises a plurality of linked orthogonal nucleobases. In some embodiments, a linked orthogonal nucleobase has a structure selected from the group consisting of:

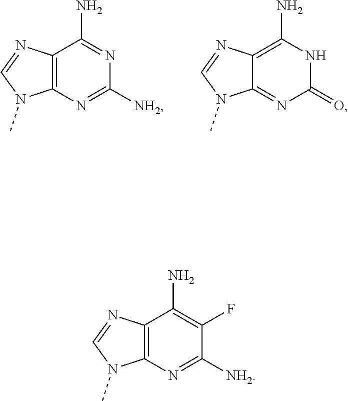

In some embodiments, “ [symbol] ” is a bond to the copy polynucleotide strand. In some embodiments, [symbol] the linked orthogonal nucleobase achieves Watson-Crick base pairing with the linked signal nucleobase.

In some embodiments, the linked signal nucleobase comprises the structure:

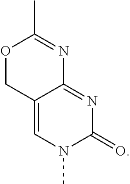

In some embodiments, the linked signal nucleobase does not achieve Watson-Crick base pairing with a natural nucleobase. In some embodiments, the linked orthogonal nucleobase does not achieve Watson-Crick base pairing with a natural nucleobase.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts several exemplary options for base pairing of newly generated base D with complimentary base R.

FIG. 2 depicts an exemplary scheme for meC-Seq DNA sample preparation procedure prior to sequencing.

FIG. 3 is a flow chart illustrating tagmentation with methylated double stranded DNA fragment binding to bead-linked transposome (BLT) for transposition, in accordance with an embodiment of the present disclosure.

FIG. 4 is a flow chart illustrating the formation of an anchor strand from a template strand, in accordance with an embodiment of the present disclosure.

FIG. 5 is a flow chart illustrating glycosylase treatment to cleave 5-methyl cytosine (5mC) from DNA duplex to generate a one-base pair gap, in accordance with an embodiment of the present disclosure.

FIG. 6 is a flow chart illustrating the selective chemical conversion of a natural nucleobase into an orthogonal nucleobase, in accordance with an embodiment of the present disclosure.

FIG. 7 is a flow chart illustrating unnatural base pair conversion chemistries by extending with standard dNTPs to generate a modified base, in accordance with an embodiment of the present disclosure.

FIG. 8 is a flow chart illustrating unnatural base pair conversion chemistries by extending with thioguanine dNTP to generate a modified base, in accordance with an embodiment of the present disclosure.

FIG. 9 is a flow chart illustrating base pair bonding and interactions with modified base, in accordance with an embodiment of the present disclosure.

FIG. 10 is a flow chart illustrating the incorporation of a signal nucleobase into a signal polynucleotide strand, in accordance with an embodiment of the present disclosure.

FIG. 11 is a flow chart illustrating six-base sequencing to generate six-base polynucleotide sequences, in accordance with an embodiment of the present disclosure.

### DETAILED DESCRIPTION

Embodiments of the present disclosure relate to methods of detecting methylation sites in a polynucleotide. In some embodiments, the methods include six-nucleobase nucleotides for use in sequencing and methylation detection applications, for example, sequencing-by-synthesis (SBS). The six-nucleobase nucleotides offer direct detection methodology that allows for detection of 5-methylcytosine and simultaneous sequencing of a full genome without loss of single nucleotide polymorphism information. Six-nucleobase SBS detection methodology is more sensitive compared to those known in the art. In particular, this methodology may be used for small amounts of analyte and/or difficult sample types, such as cell-free DNA from plasma and single-cell samples.

One method developed to avoid the shortcomings of WGBS is enzymatic methyl-seq (EM-seq, New England Biolabs). EM-seq replaces the bisulfite chemistry with sequential treatment by TET 5-methylcytosine oxidase followed by apolipoprotein B mRNA editing enzyme, catalytic polypeptide like (APOBEC), a variant of the human cytosine deaminase. TET oxidizes 5-methylcytosine (5mC) and 5-hydroxymethylcytosine (5hmC) to 5-carboxylcytosine (5caC) while APOBEC deaminates unmodified cytosine, 5-methylcytosine, and 5-hydroxymethylcytosine to uracil. EM-seq avoids many of the dropout and GC bias issues of WGBS, by eliminating the harsh bisulfite chemistry, but EM-seq still functions as an “inverse detection” assay. The 5mC and 5hmC converted to 5caC by TET are protected from deamination by APOBEC and read as cytosine during sequencing while unmodified cytosine is deaminated by APOBEC and read as thymine during sequencing. TET-assisted pyridine-borane sequencing (TAPS) uses sequential treatment by TET 5-methylcytosine oxidase followed by reduction with pyridine-borane. The reductive step converts 5caC to dihydrouracil, which is read as thymine during sequencing. TAPS only converts modified C residues and is a “direct detection” method that provides a genome that is more information-rich compared to “inverse detection” methods. However, broad adoption of TAPS is limited by the toxicity and stability of the pyridine-borane. In addition, the TET proteins required for EM-seq and TAPS can be difficult to produce at the scale needed for a commercial assay.

One embodiment is a method of detecting 5-methylcytosine nucleobases in a polynucleotide by using selective chemical methodology to convert the modified nucleobase within a polynucleotide analyte to an unnatural nucleobase. The selective chemistry produces a single, novel unnatural nucleobase (signal nucleobase) that can achieve Watson-Crick base pairing with a second unnatural partner nucleobase (orthogonal nucleobase). The pairing of the signal nucleobase and orthogonal nucleobase creates an orthogonal third base-pair from the polynucleotide analyte and a novel “six-nucleobase” alphabet.

A Sequencing-by-Synthesis (SBS) protocol using the “six-nucleobase” alphabet can then perform “six-nucleobase sequencing” to amplify and sequence to identify the 5-methylcytosine nucleobases present in the polynucleotide analyte. “Six-nucleobase sequencing” is a “direct detection” methodology that allows for detection of 5-methylcytosine and simultaneous sequencing of a full ‘four-base’ genome without loss of SNP information. This embodiment of a six-nucleobase sequencing detection methodology provides an information-rich genome and may overcome the limitations of “inverse detection” methods and can be used for detection of modified nucleobases other than 5-methylcytosine. The amplification step of SBS that preserves modification information makes the described six-nucleobase sequencing detection methodology highly sensitive, which is potentially useful for small amounts of analyte and difficult sample types such as cell-free DNA from plasma and single-cell samples. The six-nucleobase sequencing detection methodology is generally agnostic to the sequence context of the nucleobase modifications which is an advantage over alternative methylation-aware amplification methods.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the embodiments to distinguish beads in a liquid array, for example, using a microfluidic device, such as a fluorescent activated cell sorter (FACS), are described, for example, in U.S. Pat. No. 6,524,793. Further examples of arrays that can be used in the embodiments include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

The terms “blocking group” and “blocking groups” as used herein refer to any atom or group of atoms that is added to a molecule in order to prevent existing groups in the molecule from undergoing unwanted chemical reactions.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

As used herein, any “R” group(s) represent substituents that can be attached to the indicated atom. An R group may be substituted or unsubstituted. If two “R” groups are described as “together with the atoms to which they are attached” forming a ring or ring system, it means that the collective unit of the atoms, intervening bonds and the two R groups are the recited ring. For example, when the following substructure is present:

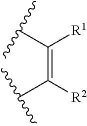

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

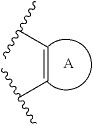

where A is an aryl ring or a carbocyclyl containing the depicted double bond.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “Cₐ to C_{b}” in which “a” and “b” are integers refer to the number of carbon atoms in an alkyl, alkenyl or alkynyl group, or the number of ring atoms of a cycloalkyl or aryl group. That is, the alkyl, the alkenyl, the alkynyl, the ring of the cycloalkyl, and ring of the aryl can contain from “a” to “b”, inclusive, carbon atoms. For example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—; a C₃ to C₄ cycloalkyl group refers to all cycloalkyl groups having from 3 to 4 carbon atoms, that is, cyclopropyl and cyclobutyl. Similarly, a “4 to 6 membered heterocyclyl” group refers to all heterocyclyl groups with 4 to 6 total ring atoms, for example, azetidine, oxetane, oxazoline, pyrrolidine, piperidine, piperazine, morpholine, and the like. If no “a” and “b” are designated with regard to an alkyl, alkenyl, alkynyl, cycloalkyl, or aryl group, the broadest range described in these definitions is to be assumed. As used herein, the term “C₁-C₆” includes C₁, C₂, C₃, C₄, C₅ and C₆, and a range defined by any of the two numbers. For example, C₁-C₆ alkyl includes C₁, C₂, C₃, C₄, C₅ and C₆ alkyl, C₂-C₆ alkyl, C₁-C₃ alkyl, etc. Similarly, C₂-C₆ alkenyl includes C₂, C₃, C₄, C₅ and C₆ alkenyl, C₂-C₅ alkenyl, C₃-C₄ alkenyl, etc.; and C₂-C₆ alkynyl includes C₂, C %, C₄, C₅ and C₆ alkynyl, C₂-C₅ alkynyl, C₃-C₄ alkynyl, etc. C₃-C₈ cycloalkyl each includes hydrocarbon ring containing 3, 4, 5, 6, 7 and 8 carbon atoms, or a range defined by any of the two numbers, such as C₃-C₇ cycloalkyl or C₅-C₆ cycloalkyl.

As used herein, “alkyl” refers to a straight or branched hydrocarbon chain that is fully saturated (e.g., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁-C₄alkyl” or similar designations. By way of example only, “C₁-C₆ alkyl” indicates that there are one to six carbon atoms in the alkyl chain, e.g., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “alkoxy” refers to the formula —OR wherein R is an alkyl as is defined above, such as “C₁-C₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “alkenyl” refers to a straight or branched hydrocarbon chain containing one or more double bonds. The alkenyl group may have 2 to 20 carbon atoms, although the present definition also covers the occurrence of the term “alkenyl” where no numerical range is designated. The alkenyl group may also be a medium size alkenyl having 2 to 9 carbon atoms. The alkenyl group could also be a lower alkenyl having 2 to 6 carbon atoms. The alkenyl group may be designated as “C₂-C₆ alkenyl” or similar designations. By way of example only, “C₂-C₆ alkenyl” indicates that there are two to six carbon atoms in the alkenyl chain, e.g., the alkenyl chain is selected from the group consisting of ethenyl, propen-1-yl, propen-2-yl, propen-3-yl, buten-1-yl, buten-2-yl, buten-3-yl, buten-4-yl, 1-methyl-propen-1-yl, 2-methyl-propen-1-yl, 1-ethyl-ethen-1-yl, 2-methyl-propen-3-yl, buta-1,3-dienyl, buta-1,2,-dienyl, and buta-1,2-dien-4-yl. Typical alkenyl groups include, but are in no way limited to, ethenyl, propenyl, butenyl, pentenyl, and hexenyl, and the like.

As used herein, “alkynyl” refers to a straight or branched hydrocarbon chain containing one or more triple bonds. The alkynyl group may have 2 to 20 carbon atoms, although the present definition also covers the occurrence of the term “alkynyl” where no numerical range is designated. The alkynyl group may also be a medium size alkynyl having 2 to 9 carbon atoms. The alkynyl group could also be a lower alkynyl having 2 to 6 carbon atoms. The alkynyl group may be designated as “C₂-C₆ alkynyl” or similar designations. By way of example only, “C₂-C₆ alkynyl” indicates that there are two to six carbon atoms in the alkynyl chain, e.g., the alkynyl chain is selected from the group consisting of ethynyl, propyn-1-yl, propyn-2-yl, butyn-1-yl, butyn-3-yl, butyn-4-yl, and 2-butynyl. Typical alkynyl groups include, but are in no way limited to, ethynyl, propynyl, butynyl, pentynyl, and hexynyl, and the like.

As used herein, “heteroalkyl” refers to a straight or branched hydrocarbon chain containing one or more heteroatoms, that is, an element other than carbon, including but not limited to, nitrogen, oxygen, and sulfur, in the chain backbone. The heteroalkyl group may have 1 to 20 carbon atoms, although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 6 carbon atoms. The heteroalkyl group may be designated as “C₁-C₆ heteroalkyl” or similar designations. The heteroalkyl group may contain one or more heteroatoms. By way of example only, “C₄-C₆ heteroalkyl” indicates that there are four to six carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (e.g., rings which share adjacent pairs of atoms) groups provided that the entire ring system is aromatic.

As used herein, “aryl” refers to an aromatic ring or ring system (e.g., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆-C₁₀ aryl,” “C₆ or C₁₀ aryl,” or similar designations. Examples of aryl groups include, but are not limited to, phenyl, naphthyl, azulenyl, and anthracenyl.

An “aralkyl” or “arylalkyl” is an aryl group connected, as a substituent, via an alkylene group, such as “C₇₋₁₄ aralkyl” and the like, including but not limited to benzyl, 2-phenylethyl, 3-phenylpropyl, and naphthylalkyl. In some cases, the alkylene group is a lower alkylene group (e.g., a C₁-C₆ alkylene group).

As used herein, “heteroaryl” refers to an aromatic ring or ring system (e.g., two or more fused rings that share two adjacent atoms) that contain(s) one or more heteroatoms, that is, an element other than carbon, including but not limited to, nitrogen, oxygen, and sulfur, in the ring backbone. When the heteroaryl is a ring system, every ring in the system is aromatic. The heteroaryl group may have 5-18 ring members (for example, the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (e.g., a C₁-C₆ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃-C₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

As used herein, “cycloalkyl” means a fully saturated carbocyclyl ring or ring system. Examples include cyclopropyl, cyclobutyl, cyclopentyl, and cyclohexyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (e.g., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

An “O-carboxy” group refers to a “—OC(═O)R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-carboxy” group refers to a “—C(═O)OR” group in which R is selected from the group consisting of hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes carboxyl (e.g., —C(═O)OH).

An “alkyl C-carboxy” group refers to an “—(CH)ₙC(═O)OR” group in which n is from 1 to 6 and the C(═O)OR group is the same as defined for a “C-carboxy” group.

A “thioalkyl” group refers to an “—SR” group in which R is selected from C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfonyl” group refers to an “—SO₂R” group in which R is selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “sulfino” group refers to a “—S(═O)OH” group.

A “S-sulfonamido” group refers to a “—SO₂NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-sulfonamido” group refers to a “—N(RA)SO₂R_{B}” group in which R_{A} and R_{b} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

A “C-amido” group refers to a “—C(═O)NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “N-amido” group refers to a “—N(R_{A})C(═O)R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein.

An “amino” group refers to a “—NR_{A}R_{B}” group in which R_{A} and R_{B} are each independently selected from hydrogen, C₁-C₆ alkyl, C₂-C₆ alkenyl, C₂-C₆ alkynyl, C₃-C₇ carbocyclyl, C₆-C₁₀ aryl, 5-10 membered heteroaryl, and 3-10 membered heterocyclyl, as defined herein. A non-limiting example includes free amino (e.g., —NH₂).

An “aminoalkyl” group refers to an amino group connected via an alkylene group.

An “alkoxyalkyl” group refers to an alkoxy group connected via an alkylene group, such as a “C₂-C₈ alkoxyalkyl” and the like.

An “aralkoxy” or “arylalkoxy” is an aryl group connected, as a substituent, via an alkoxy group, such as “C₇-14 arylalkoxy” and the like, including but not limited to benzyl, 2-phenylethyl, 3-phenylpropyl, and naphthylalkyl. In some cases, the alkoxy group is a lower alkoxy group (e.g., a C₁-C₃ alkoxy group).

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₁-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, —CN, hydroxy, C₁-C₆ alkoxy, C₁-C₆ alkoxy(C₁-C₆)alkyl (e.g., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, —SO₃H, sulfino, —OSO₂C₁₋₄alkyl, and oxo (═O). Wherever a group is described as “optionally substituted” that group can be substituted with the above substituents.

The term “hydroxy” as used herein refers to a —OH group.

The term “cyano” group as used herein refers to a “—CN” group.

The term “diazo” as used herein refers to a —N₂ group.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, for example, a sugar lacking a hydroxyl group that is present in ribose. The nitrogen containing heterocyclic base can be purine or pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleotide is also a phosphate ester of a nucleoside, with esterification occurring on the hydroxy group attached to the C-3 or C-5 of the sugar. Nucleotides are usually mono, di- or triphosphates.

As used herein, a “nucleoside” is structurally similar to a nucleotide, but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art, and includes its tautomers. Similarly, the term “pyrimidine base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

The term “nucleobase” as used herein, is a purine base or a pyrimidine base. Non-limiting examples of purine nucleobases include adenine (A), guanine (G), and derivatives or analogs thereof. Non-limiting examples of pyrimidine nucleobases include cytosine (C), thymine (T), uracil (U), and derivatives or analogs thereof.

The term “Watson-Crick base pairing” as used herein, is the complementary pattern of hydrogen bonding achieved between two nucleobases (e.g., guanine-cytosine and adenine-thymine) of opposite polynucleotide strands. The pattern of hydrogen bonding is predictable and reliable and allows double-stranded polynucleotide strands (e.g., the DNA double-helix), to maintain a regular helical structure that is subtly dependent on its nucleotide sequence.

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, “derivative” or “analogue” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

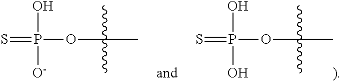

As used herein, the terms “monophosphate,” “diphosphate,” and “triphosphate” are used in their ordinary sense as understood by those skilled in the art, and include protonated forms.
Method of detecting 5-methylcytosine

In the human genome, the most prevalent modified base is 5-methyl cytosine (5mC), which accounts for ˜1% of all nucleobases. Detection of 5mC is an area of importance for understanding epigenetic markers that may be implicated in cancer, diabetes, and other diseases.

There are several methods developed to map DNA methylation events including bisulfite sequencing, EM-seq (NEB) and TAPS (Base Genomics). However, all these methods rely on selectively converting C or 5mC and its derivative to U or its derivatives using chemical or enzymatic reactions. Therefore, the DNA samples must be sequenced twice: first C/5mC and derivatives are read as C; then after chemical/enzymatic conversion, C/5mC and the derivatives are read as T. The time and cost associated with 5mC-sequencing are double of the standard sequencing, with a concomitant loss of efficiency of conversion of the 5mC and its derivatives, and potential DNA damage by exposing to the hard chemical reagents.

Accordingly, embodiments provided herein relate to methods for detection and/or recognition of 5mC and/or its derivatives. In some embodiments, the methods include detecting a new artificial base directly by sequencing. In some embodiments, a third base pair in addition to A-T, G-C base pairs is used to facilitate 5mC recognition.

Several groups have worked on developing expanding “genetic alphabets” including Romesberg, Hirao, and Benner by introducing unnatural base pair (UBP) to expand the genetic coding system. Current Opinion in Biotechnology 2018, 51:8-15. Benner's group introduced P-Z pair with a different hydrogen bonding donor and acceptor pattern from those of the natural base pairs. In contrast, Romesberg's group synthesized a series of hydrophobic base analogues, such as 5SICS-NaM and TPT3-NaM. Hirao's group developed the hydrophobic Ds-Px pair by the concept of shape complementarity with steric and electrostatic exclusions. These UBPs exhibit high fidelity in replication and/or transcription and demonstrated various applications using the UBPs.

Some embodiments of the methods provided herein relate to generating a third base pair by altering hydrogen bonding donor-acceptor pattern, thereby forming base pair exclusively with mC and its derivatives. In some embodiments, the methods include polymerase acceptance of UBP. Without being bound by theory, all four standard nucleotides (A, C, G, and T) present electron density to the minor groove, either from N3 of the purines or from the exocyclic oxygen of the pyrimidines, and polymerases seek this electron density as a way of achieving uniform acceptance of their four substrates.

In some embodiments, the methods include converting meC is converted to hmC using TET enzyme. In some embodiments, the methods further include treatment of hydroxy and exo-amino group on hmC with acid chloride, resulting in a six member oxazine ring. In some embodiments, the six-membered oxazine ring alters hydrogen bonding pattern of C (D, A, A) to a new base R (A, A, A), as shown in the following scheme:

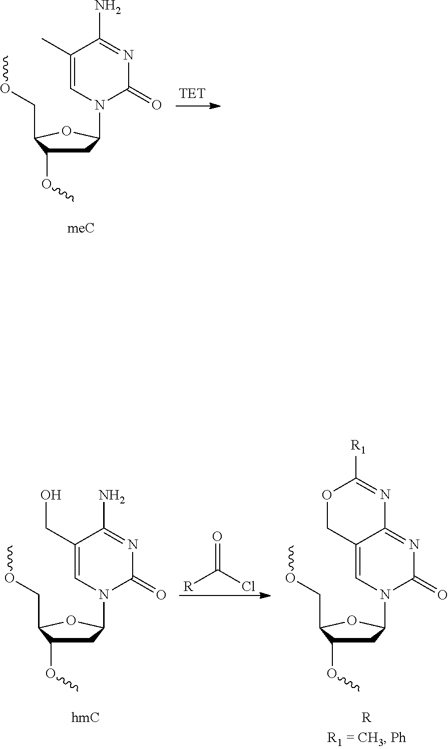

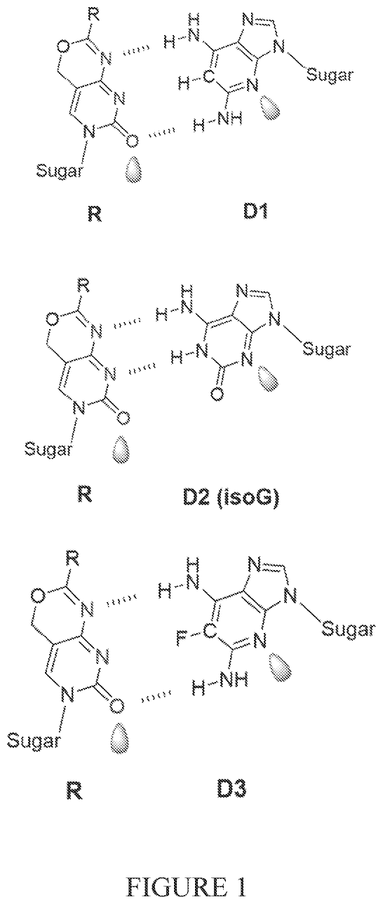

In some embodiments, the base complimentary to base R meets the basic the Watson-Crick geometry requirement: a small pyrimidine analogue with one ring complements in size a large purine analogue with two rings, joined by two or three hydrogen bonds. In some embodiments, the new base (D) is complementary to R as shown in FIG. 1.

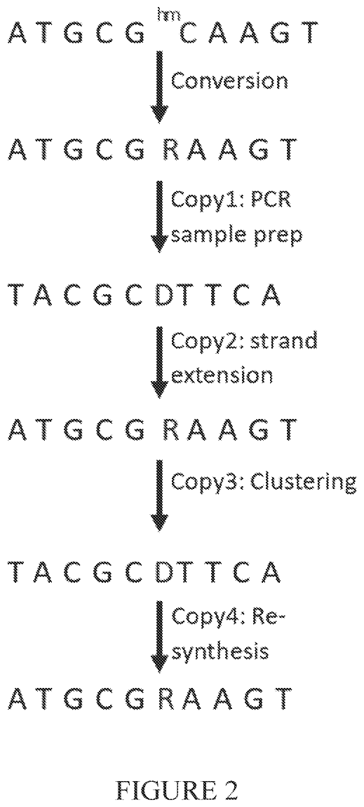

In some embodiments, the DNA samples go through several replication events before ready for SBS on the surface, and prior to sequencing. In some embodiments, the third base pair is copied over together with A-T & G-C as shown in FIG. 2. As shown in FIG. 2, hmC is converted to R, followed by stand PCR enrichment, strand extension and clustering, and the R-D pair is copied between each other together with A-T & G-C base pairs.

In some embodiments, the SBS sequencing, the corresponding fully functional nucleotides (ffNs) are constructed in the same fashion of the standard ffNs in SBS sequencing. Because only one of the bases R or D appears in the strands on the cluster for sequence, the same detection method can be applied, such as using ffN-dye or secondary labelling of ffN-substrate+dye-protein.

Some embodiments of the present disclosure relate to methods of detecting a modified nucleobase in a target polynucleotide strand. Particular embodiments relate to methods of detecting 5-methylcytosine in a target polynucleotide strand.

In some embodiments, the methods include providing a target polynucleotide strand. The target polynucleotide strand comprises a polynucleotide or an oligonucleotide. In some embodiments, the target polynucleotide strand comprises a DNA strand or an RNA stand. The target polynucleotide strand includes at least one modified nucleobase. In some embodiments, the target polynucleotide strand includes a plurality of modified nucleobases. As used herein, the term “modified nucleobase” is a nucleobase having a structural variation when compared to a naturally occurring nucleobase. In some embodiments, the structural variation is the result of a chemical transformation including alkylation, acetylation, an acid-base reaction, reduction, oxidation, and combinations of any of the foregoing.

The methods include forming a copy polynucleotide strand. In some embodiments, the copy polynucleotide strand is a growing copy polynucleotide strand. The copy polynucleotide strand is complementary to at least a portion of the target polynucleotide strand. The copy polynucleotide strand includes at least one paired nucleobase. In some embodiments, the copy polynucleotide strand includes a plurality of paired nucleobases. As used herein, the term “paired nucleobase” is a nucleobase capable of undergoing Watson-Crick base pairing with the modified nucleobase.

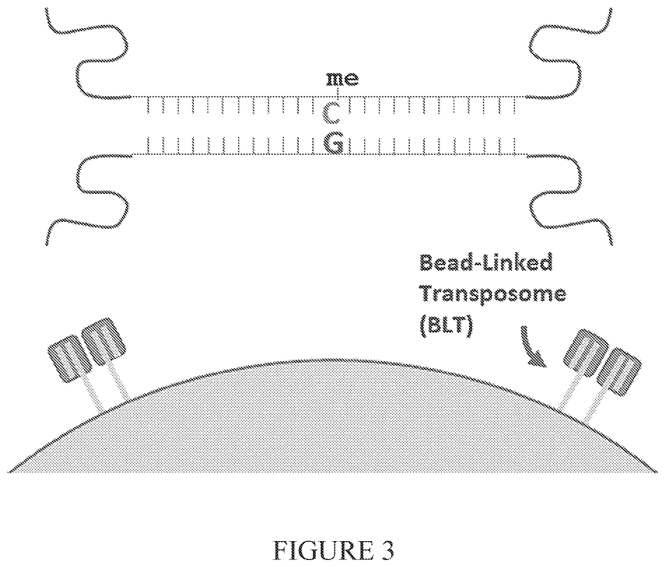

Exemplary steps for performing methods of six-base sequencing and amplification are described in the accompanying drawings. The methods provided herein and exemplified in the accompanying drawings are intended to be illustrative, and additional embodiments are provided as described throughout the specification and as understood in view the detailed description. In some embodiments, the methods include tagmentation of modified DNA (FIG. 3). As shown in FIG. 3, double-stranded target DNA (dsDNA) containing the target modification (e.g., a modified nucleobase), is tagmented using a bead-linked transposome (BLT) system, covalently linking the 5′ end of each strand in the target fragment to a magnetic bead. This is followed by a gap-fill ligation step, in which the transposase and the non-transfer strand of the transposon are washed away, and an adapter sequence is attached to the 3′ end of the non-transfer strand.

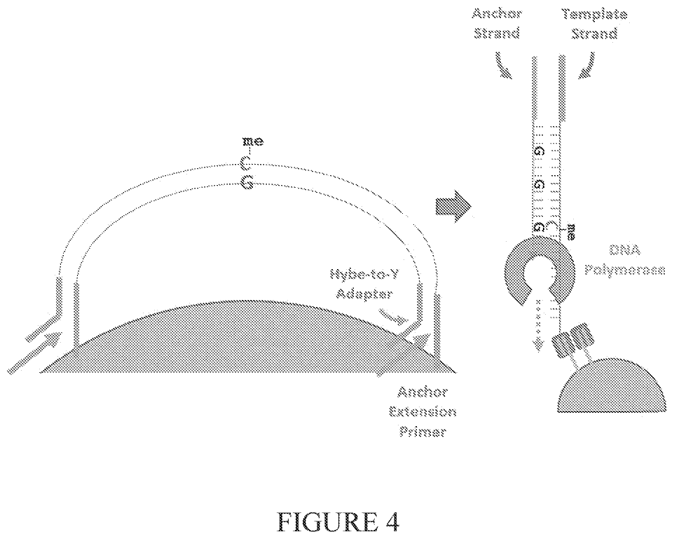

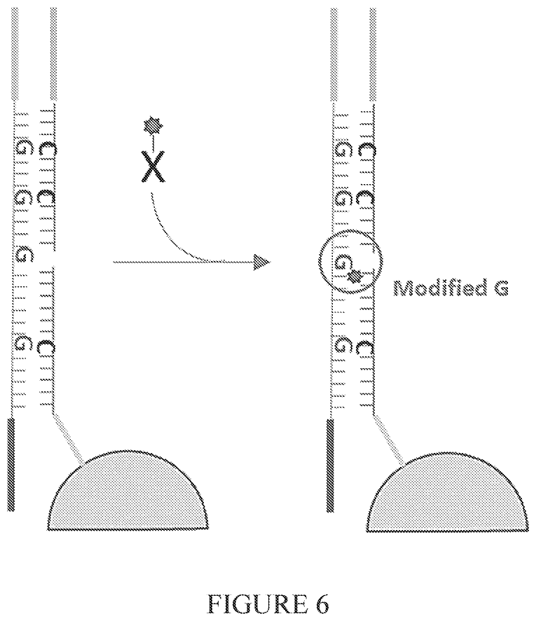

In some embodiments, the methods include synthesis of an anchor strand (FIG. 4). A primer is hybridized to the free 3′ end of the adapter attached in FIG. 3. A strand-displacing polymerase is then used to synthesize a complementary strand. This causes the two original template strands to separate, leaving two dsDNA fragments each with one 5′ end attached to the bead. The anchor strand serves two purposes; first, it provides a uniformly non-modified strand to allow any short fragments remaining after glycosylase treatment to remain bound to the bead, and second, it allows for the introduction of thioguanine residues if a nucleophilic aromatic substitution chemistry is used for G conversion (FIG. 6). As shown in FIG. 4, the target polynucleotide strand is shown (template strand). Not wishing to be limited and solely for the purpose of illustration, the modified nucleobase is represented as a methylated cytosine in FIG. 4. The copy nucleotide strand (anchor strand) is formed by sequential addition of nucleotides to the copy nucleotide strand in the 5′ to 3′ direction by the polymerase to form the copy nucleotide strand complementary to the target polynucleotide strand. In some embodiments, one or more of the nucleotides added to the copy nucleotide strand includes the paired nucleobase. The paired nucleobase of the copy nucleotide strand achieves Watson-Crick base pairing with the modified nucleobase of the target polynucleotide strand. Not wishing to be limited and solely for the purpose of illustration, the polymerase is represented as DNA polymerase and the paired nucleobase is represented as guanine in FIG. 4.

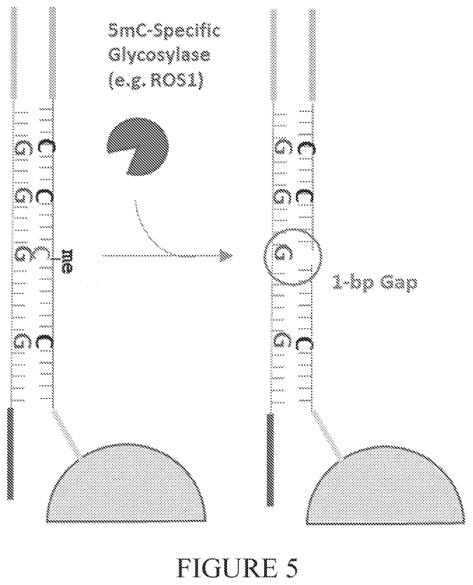

In some embodiments, the methods include removing the at least one modified nucleobase, or the plurality of modified nucleobases, from the target polynucleotide strand. As shown in FIG. 5, the anchor strand—template duplex DNAs are treated with a DNA glycosylase that specifically targets the modification of interest. This exposes the Watson-Crick-Franklin (WCF) face of the anchor strand base opposite the modified base for chemical transformation in FIG. 6. DNA glycosylases can have two different enzymatic mechanisms: ‘monofunctional’ glycosylases cleave only the N-glycosidic bond connecting the base to the backbone (deoxy)ribose, leaving an abasic site with the backbone sugar and phosphate intact, while ‘bifunctional’ glycosylases both remove the base and cleave the nucleic acid backbone. Either type could be used in this step, although a monofunctional glycosylase would have the added benefit of retaining a covalent linkage throughout the template following base cleavage. This would prevent dissociation of the template strand in cases where many modifications lie close together on a single fragment. Bifunctional glycosylases targeting 5mC are known to exist in nature, with the best characterized example being the ROS1 glycosylase from Arabidopsis.

Tn some embodiments, engineered or natural glycosylases targeting other modifications may be used, enabling six-base detection of these modifications as well. Accordingly, in some embodiments, removing the modified nucleobase forms a gapped polynucleotide strand. In some embodiments, the gapped polynucleotide strand includes an anucelobasic site (1-bp Gap). As used herein, the term “anucelobasic site” is a location of a polynucleotide strand where a nucleobase is not attached to the sugar-phosphate backbone. In other words, the anucelobasic site is absent an N-glycosidic bond to the sugar-phosphate backbone of the polynucleotide strand. In some embodiments, the anucelobasic site is an apurinic site or apyrimidinic site. As used herein, the terms “apurinic site” and “apyrimidinic site” refer to a location of a polynucleotide strand where a purine or pyrimidine, respectively, is not attached to the sugar-phosphate backbone of the polynucleotide strand. In some embodiments, the anucelobasic site is an inadeninic site, incytosinic site, inguaninic site, inthyminic site, or inuracilic site. As used herein, the terms “inadeninic site”, “incytosinic site”, “inguaninic site”, “inthyminic site”, and “inuracilic site” refer to a location of a polynucleotide strand where an adenine, cytosine, guanine, thymine, or uracil, respectively, is not attached to the sugar-phosphate backbone of the polynucleotide strand.

In some embodiments, the methods include converting the paired nucleobase into the orthogonal nucleobase, or converting the plurality of paired nucleobases into a plurality of orthogonal nucleobases (FIG. 6). In some embodiments, the methods include chemical transformation of exposed DNA bases to introduce a third DNA base-pair. In some embodiments, following glycosylase treatment, modified nucleobases are converted to either an apurinic/apyrimidinic (AP site) or a 1-bp gap in the template sequence. In either case, the base-pairing face of the anchor strand nucleobase opposite the cleaved modification site may be exposed to solvent. In some embodiments, the modified duplex is treated with a small molecule reagent that selectively installs a functional group on the exposed base, such as guanine in the case of 5mC. In some embodiments, this functional group disrupts base-pairing with both the standard WCF partner and the other three natural DNA bases and selectively base-pairs with an unnatural base partner to form a third DNA base.

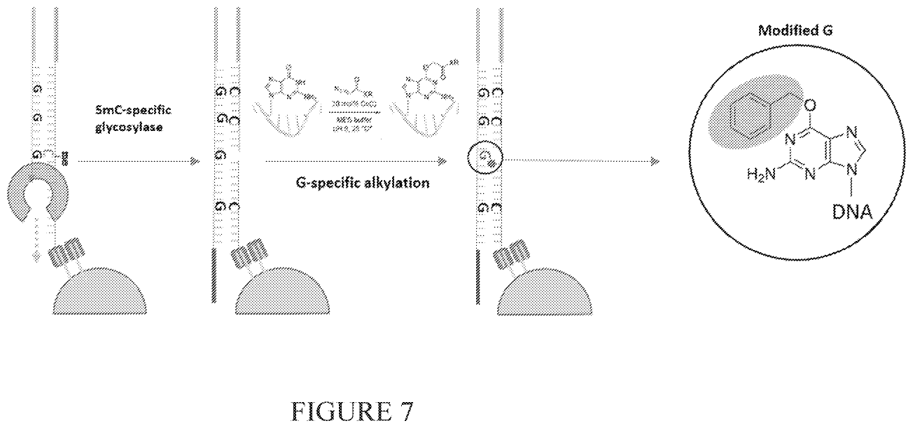

In some embodiments, the formation of a third DNA base is achieved as shown in FIG. 7, wherein standard nucleobases are used for synthesis of the anchor strand, and exposed G bases are modified using a G-specific alkylating agent. In some embodiments, a family of diazocarbonyl compounds that give highly regioselective alkylation of the 06 position of guanine and inosine via a copper(I)-carbene intermediate in ssDNA is used to install a bulky hydrophobic group at guanine O6 that may change the base-pairing properties of the modified nucleobase by steric blocking. In some embodiments, orthogonal base-pairing is achieved using a partner unnatural nucleobase that maintains the H-bonds to the extracyclic amine of G while forming a hydrophobic interaction with the blocking group.

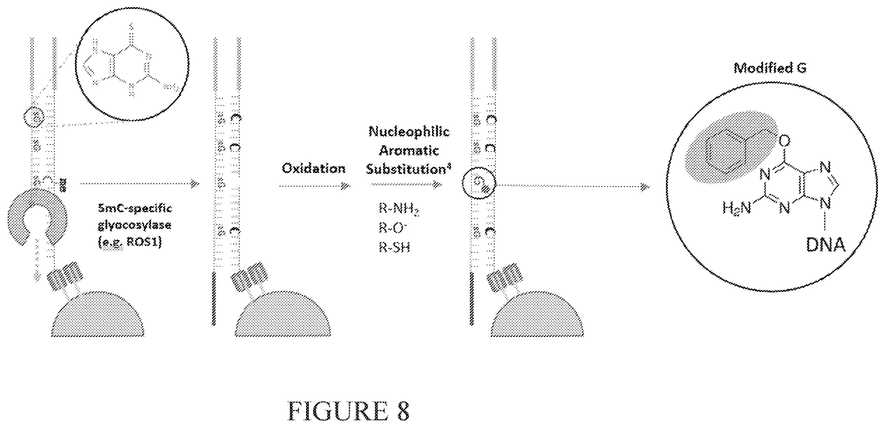

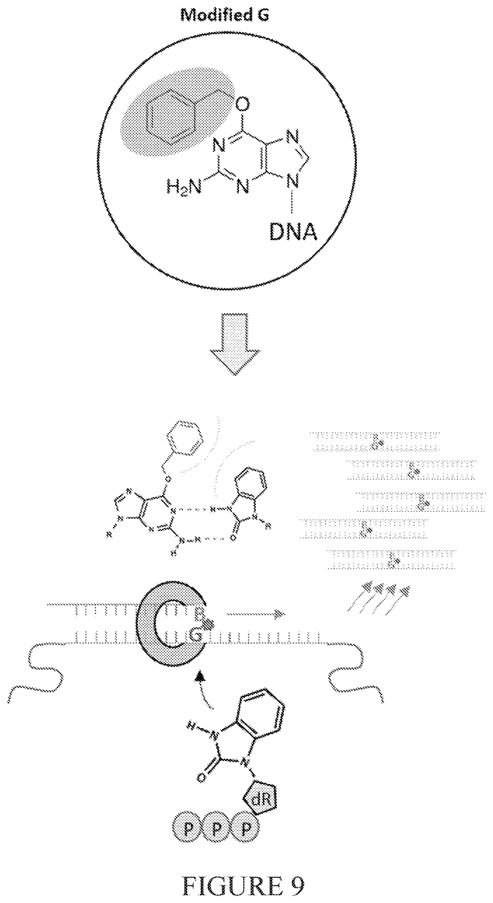

In some embodiments, the formation of a third DNA base is achieved as shown in FIG. 8, wherein alternative transformation chemistry based on aromatic nucleophilic substitution in RNA pulse-chase experiments are used. In some embodiments, the strand to be modified has 6-thioguanine substituted for guanine, which may include the use of a 6-thioguanine dNTP during synthesis of the anchor strand, as shown in FIG. 8. In some embodiments, oxidation of the S6 atom of thioguanine generates sulfonate, which can act as a leaving group for aromatic substitution by sulfur, oxygen, or nitrogen nucleophiles. In some embodiments, an O-, S- or N-linked benzyl group is inserted at the 6 position to generate an analog of O-benzylguanine (BnG). In some embodiments, the generated nucleobase is capable of orthogonal base-pairing with unnatural bases such as the “Benzi” nucleobase (FIG. 9).

In some embodiments, the chemical conversion shown in FIG. 6 includes subjecting the paired nucleobase to a transformation process selected from an enzymatic process, a chemical process, a thermal process, an irradiation process, or any combination of the foregoing. In some embodiments, the paired nucleobase is converted with a chemical process. In some embodiments, the chemical process includes alkylation, acetylation, cycloaddition, elimination, isomerization, oxidation, reduction, substitution, or combinations of any of the foregoing. The anucelobasic site of the gapped polynucleotide strand decreases the steric bulk around the paired nucleobase that exposes the paired nucleobase and facilitates the transformation of the paired nucleobase. For example, chemical reagents can access the paired nucleobase more easily as a result of the decreased steric bulk around the paired nucleobase.

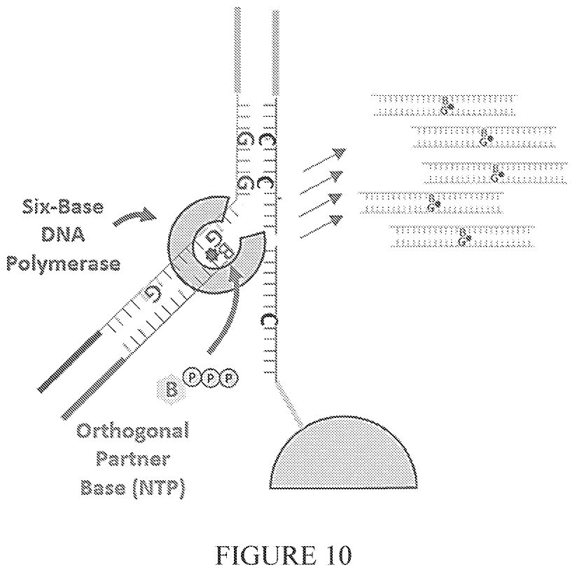

In some embodiments, the methods include incorporating at least one signal nucleobase into the signal polynucleotide strand. In some embodiments, the methods include incorporating a plurality of signal nucleobases into the signal polynucleotide strand. In some embodiments, the signal polynucleotide strand is a growing signal polynucleotide strand. The signal polynucleotide strand is complementary to at least a portion of the copy polynucleotide strand. Referring to FIG. 10, incorporation of the signal nucleobase into the signal polynucleotide strand by a polymerase is illustrated therein. The signal nucleotide strand is formed by sequential addition of nucleotides to the signal nucleotide strand in the 3′ to 5′ direction using a polymerase to form the signal nucleotide strand complementary to the copy polynucleotide strand. In some embodiments, the polymerase is a six-base DNA polymerase. In some embodiments, one or more of the nucleotides added to the signal nucleotide strand includes the signal nucleobase. The signal nucleobase of the signal polynucleotide strand achieves Watson-Crick base pairing with the orthogonal nucleobase of the copy nucleotide strand and thereby creates a third DNA base pair. The signal nucleotide includes a detectable label, as described elsewhere herein. The identity of a newly incorporated signal nucleotide is determined with the detectable label and allows for detection of the modified nucleobase in the target polynucleotide strand. The identity of the signal nucleobase corresponds to the identity of the modified nucleobase because of Watson-Crick base pairing between the modified nucleobase and the paired nucleobase, the orthogonal nucleobase occupies the same position in the copy polynucleotide strand as the paired nucleobase, and Watson-Crick base pairing between the orthogonal nucleobase and the signal nucleobase. In other words, detecting the modified nucleobase in the target polynucleotide strand is accomplished with the detectable label of the newly incorporated signal nucleotide.

In some embodiments, following chemical conversion, the anchor strand contains the orthogonal base-pair mark opposite the abasic sites generated in FIG. 5 and is attached to the bead through hybridization to the fragmented template strand. After washing away the conversion agent, the anchor strand is eluted from the bead by denaturation, and amplified using a DNA polymerase and a dNTP mixture containing the triphosphate of the unnatural partner base. By way of example, in the case of the BnG/Benzi system, a mutated KlenTaq polymerase was used to avoid stalling at the BnG adduct and enhance specific incorporation of Benzi.

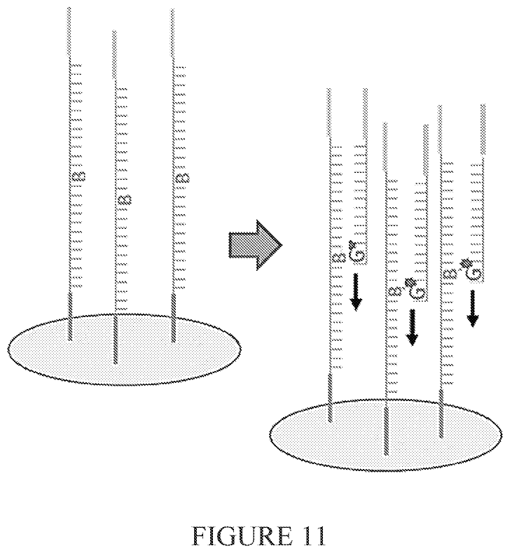

In some embodiments, the methods include six-base sequencing, as shown in FIG. 11. In some embodiments, amplification produces double-stranded DNA six-base polynucleotides. In some embodiments, sequencing of the six-base polynucleotides is performed with an extended SBS chemistry that includes additional fully functional nucleotides (FFNs) for the two unnatural bases, as well as an engineered sequencing polymerase that can tolerate these modifications.

In some embodiments, the signal nucleobase comprises a structure selected from the group consisting of:

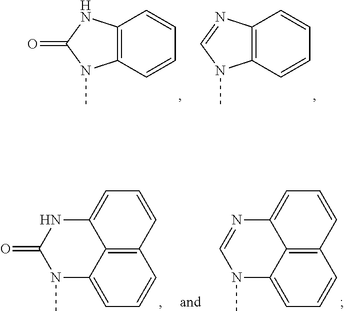

wherein “ [symbol] ” is a bond to the signal polynucleotide strand.

[symbol]

In some embodiments, the signal nucleobase comprises the structure:

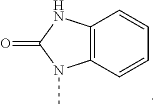

In some embodiments, the signal nucleobase does not achieve Watson-Crick base pairing with a linked orthogonal nucleobase or a natural nucleobase. The term “natural nucleobase” as used herein, includes adenine (A), guanine (G), cytosine (C), thymine (T), and uracil (U).

In some embodiments, the orthogonal nucleobase has the structure selected from:

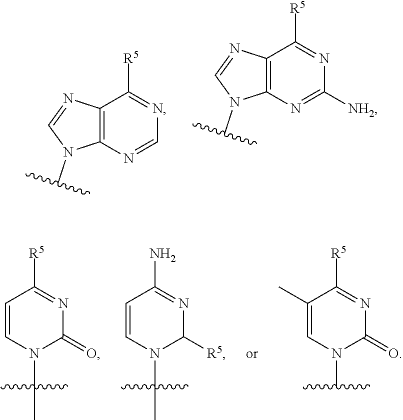

In some embodiments, R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, R⁵ is selected from optionally substituted C₁-C₃ alkyl-C-carboxy or optionally substituted C₇-C₁₂ aralkyl. In some embodiments, R⁵ is CH₂C(O)OR³ and R³ is methyl, ethyl, or t-butyl. In some embodiments, R⁵ is CH₂C(O)OEt. In some embodiments, R⁵ is NHPh, OPh, SPh, NHCH₂Ph, OCH₂Ph, or SCH₂Ph. In some embodiments, R⁵ is OCH₂Ph. In some embodiments, R⁵ is phenyl or benzyl. In some embodiments, the orthogonal nucleobase is O-benzylguanine. In some embodiments, the orthogonal nucleobase may comprise a functional group selected from hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, C₅-C₁₂ heteroaralkyl, and optionally substituted derivatives of any of the foregoing. In some embodiments, the functional group is optionally substituted C₁-C₃ alkyl-C-carboxy, optionally substituted C₇-C₁₂ aralkyl, or optionally substituted C₇-C₁₂ arylalkoxy. The orthogonal nucleobase may achieve Watson-Crick base pairing with the signal nucleobase. The functional group on the orthogonal nucleobase allows the orthogonal nucleobase to achieve Watson-Crick base pairing selectively with the structural features of the signal nucleobase and form a third DNA base pair. The third DNA base pair creates the six-nucleobase polynucleotide. The functional group on the orthogonal nucleobase prevents Watson-Crick base pairing with a natural nucleobase. In some embodiments, the orthogonal nucleobase does not achieve Watson-Crick base pairing with a linked signal nucleobase or a natural nucleobase.

In some embodiments, the modified nucleobase is selected from the group consisting of modified adenine, modified cytosine, modified guanine, modified thymine, and modified uracil. In some embodiments, the modified nucleobase is an acetylated nucleobase or an alkylated nucleobase. In some embodiments, the modified nucleobase is a C₁-C₆ alkylated nucleobase. In some embodiments, the modified nucleobase is selected from C₁-C₆ alkylated adenine, C₁-C₆ alkylated cytosine, C₁-C₆ alkylated guanine, C₁-C₆ alkylated thymine, and C₁-C₆ alkylated uracil. In some embodiments, the modified nucleobase is a methylated nucleobase. In some embodiments, the modified nucleobase is selected from methylated adenine, methylated cytosine, methylated guanine, methylated thymine, and methylated uracil. In some embodiments, the modified nucleobase is selected from 2-methyladenine, 8-methyladenine, 5-methylcytosine, 6-methylcytosine, 8-methylguanine, 6-methylthymine, or any combination of the foregoing. In some embodiments, the modified nucleobase is 5-methylcytosine.

In some embodiments, the paired nucleobase is selected from the group consisting of adenine, cytosine, guanine, thymine, and uracil. In some embodiments, the paired nucleobase is guanine.

The method includes removing the modified nucleobase from the target polynucleotide strand. In some embodiments, removing is accomplished by a glycosylase. In some embodiments, the glycosylase removes the modified nucleobase from the target polynucleotide strand to form the gapped polynucleotide strand as shown in FIG. 5. The glycosylase is configured to recognize the structure of the modified nucleobase and facilitate its removal. In some embodiments, the glycosylase is capable of hydrolyzing covalent bonds present in N-glycosyl compounds, O-glycosyl compounds, S-glycosyl compounds, or any combination of the foregoing. In some embodiments, the glycosylase is a naturally occurring glycosylase or a rationally engineered glycosylase. In some embodiments, the glycosylase is a naturally occurring glycosylase comprising a DNA glycosylase.

In some embodiments, the glycosylase is a monofunctional glycosylase or a bifunctional glycosylase. In some embodiments, the glycosylase is a monofunctional glycosylase. As used herein, the term “monofunctional glycosylase” is a glycosylase that cleaves the N-glycosidic bond between a nucleobase and a polynucleotide strand and does not cleave the sugar-phosphate backbone of the polynucleotide strand. In some embodiments, the monofunctional glycosylase cleaves the N-glycosidic bond between the modified nucleobase and the target polynucleotide strand and does not cleave the sugar-phosphate backbone of the target polynucleotide strand. In some embodiments, the monofunctional glycosylase creates an anucelobasic site in the target polynucleotide strand. In some embodiments, the monofunctional glycosylase creates an inadeninic site, incytosinic site, inguaninic site, inthyminic site, or inuracilic site in the target polynucleotide strand. In some embodiments, the monofunctional glycosylase creates an incytosinic site in the target polynucleotide strand. In some embodiments, the glycosylase is a bifunctional glycosylase. As used herein, the term “bifunctional glycosylase” is a glycosylase that cleaves the N-glycosidic bond between a nucleobase and a polynucleotide strand as well as the sugar-phosphate backbone of the polynucleotide strand. In some embodiments, the bifunctional glycosylase cleaves, at least, the sugar-phosphate backbone of the polynucleotide strand. In some embodiments, the bifunctional glycosylase cleaves the N-glycosidic bond between the modified nucleobase and the target polynucleotide strand as well as the sugar-phosphate backbone of the target polynucleotide strand. In some embodiments, the bifunctional glycosylase cleaves, at least, the sugar-phosphate backbone of the target polynucleotide strand.

In some embodiments, the glycosylase is a glycosylase derived from a plant source. In some embodiments, the glycosylase is a glycosylase derived from a plant that is defective in histone deacetylase activity or a plant that overexpresses histone deacetylase. In some embodiments, the glycosylase is a glycosylase derived from a plant that is insensitive to abscisic acid or a plant that is hypersensitive to abscisic acid. In some embodiments, the glycosylase is a glycosylase derived from Arabidopsis. In some embodiments, the glycosylase is a DNA glycosylase selected from the group including REPRESSOR OF SILENCING 1 (ROS1), DEMETER (DME), DEMETER-LIKE 2 (DML2), and DML3, as described in Choi et al., “DEMETER, a DNA glycosylase domain protein, is required for endosperm gene imprinting and seed viability in Arabidopsis”, 2002, Cell, 110, 33-42; and Penterman et al., “DNA demethylation in the Arabidopsis genome”, 2007, PNAS USA, 104, 6752-6757. In some embodiments, the glycosylase is ROS1 DNA glycosylase.

In some embodiments, the gapped polynucleotide strand includes one or more discontinuities in a sugar-phosphate backbone of the gapped polynucleotide strand. In some embodiments, the discontinuity is an absence of a covalent bond, a sugar, or a phosphate in the sugar-phosphate backbone. In some embodiments, the discontinuity is an absence of a covalent bond in the sugar-phosphate backbone. In some embodiments, the discontinuity is an absence of a sugar in the sugar-phosphate backbone. In some embodiments, the discontinuity is an absence of a phosphate in the sugar-phosphate backbone.

Some embodiments include converting the paired nucleobase with chemical reagents, as illustrated in FIG. 6. Not wishing to be limited and solely for the purpose of illustration, the paired nucleobase is represented as guanine in FIG. 6. In some embodiments, the chemical reagents include chemical reagents capable to perform alkylation, acetylation, cycloaddition, elimination, isomerization, oxidation, reduction, substitution, or combinations of any of the foregoing. In some embodiments, the chemical reagents include alkylating agents, oxidizing agents, nucleophiles, or combinations of any of the foregoing.

In some embodiments, the chemical reagents include a diazo compound having the structure N₂CWZ, wherein W is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, or an optionally substituted derivative of any of the foregoing: Z is selected from C(O)NR¹R², C(O)OR¹, C(O)SR¹, C(S)OR¹, and C(S)SR¹; and R¹ and R² are independently selected from C₁-C₁₂ alkyl, C₂-C₁₂ alkenyl, C₂-C₁₂ alkynyl, C₁-C₁₂ alkoxy, C₁-C₁₂ heteroalkyl, cyano, halo, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, C₁-C₁₂ thioalkyl, C₁-C₁₂ sulfonyl, or an optionally substituted derivative of any of the foregoing, and wherein R¹ and R² together optionally are 3-10 membered heterocyclyl or 5-10 membered heteroaryl. In some embodiments, the diazo compound has the structure N₂CHC(O)OR¹ and R¹ is selected from C₁-C₆ alkyl, C₂-C₆ alkenyl, C₂-C₆ alkynyl, C₁-C₆ alkoxy, C₁-C₈ heteroalkyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, C₁-C₆ thioalkyl, and C₁-C₁₂ sulfonyl. In some embodiments, the diazo compound has the structure N₂CHC(O)OR¹ and R¹ is selected from C₁-C₆ alkyl, for example methyl, ethyl, propyl, or t-butyl. In some embodiments, the diazo compound has the structure N₂CHC(O)NR¹R² and R¹ and R² are independently selected from C₁-C₁₂ alkyl, C₂-C₁₂ alkenyl, C₂-C₁₂ alkynyl, C₁-C₈ heteroalkyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and wherein R¹ and R² together optionally are 3-10 membered heterocyclyl or 5-10 membered heteroaryl. In some embodiments, the diazo compound has the structure N₂CHC(O)NR¹R² and R¹ and R² are independently selected from C₁-C₁₂ alkyl, C₂-C₁₂ alkenyl and C₂-C₁₂ alkynyl. In some embodiments, the diazo compound has the structure N₂CHC(O)NR¹R² and R¹ and R² together are 5-8 membered heterocyclyl or 5-8 membered heteroaryl.

In some embodiments, the chemical reagents include a metal catalyst. In some embodiments, the metal catalyst is an inorganic salt comprising a transition metal. In some embodiments, the transition metal is selected from Ag, Au, Co, Cu, Ir, Ni, Rh, Pd, Pt, Zn, and combinations of any of the foregoing. In some embodiments, the transition metal is selected from Ag, Cu, Ni, and Zn. In some embodiments, the transition metal is Cu. In some embodiments, the metal catalyst is an inorganic salt comprising a counterion selected from carbonate, halide, oxide, nitrate, nitrite, phosphate, sulfate, sulfide, sulfite, and combinations of any of the foregoing. In some embodiments, the counterion is chloride, iodide, sulfate. In some embodiments, the metal catalyst is copper chloride, copper iodide, copper sulfate, and combinations of any of the foregoing. In some embodiments, the metal catalyst is copper chloride. In some embodiments, the metal catalyst is copper iodide. In some embodiments, the metal catalyst is copper sulfate. In some embodiments, the metal catalyst includes a ligand. In some embodiments, the ligand comprises an optionally substituted 3-6 membered heterocycle. In some embodiments, the ligand comprises a 3-6 membered heterocycle substituted with one or more groups selected from C₁-C₁₂ alkyl, C₂-C₁₂ alkenyl, C₂-C₁₂ alkynyl, C₁-C₁₂ alkoxy, C₁-C₁₂ heteroalkyl, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl. In some embodiments, the ligand comprises a C₆-C₁₂ aryl-substituted 3-6 membered N-containing heterocyclic carbene. In some embodiments, the ligand is mesitylimidazolinium. In some embodiments, the metal catalyst is mesitylimidazolinium copper chloride (MesCuCl). In some embodiments, the chemical reagents include one or more reducing agents. In some embodiments, the reducing agent is an inorganic salt. In some embodiments, the reducing agent comprises ascorbate, formate, oxalate, peroxide, phosphite, thiosulfate, and combinations of any of the foregoing. In some embodiments, the reducing agent comprises ascorbate. In some embodiments, the chemical reagents include the diazo compound, the metal catalyst, and the reducing agent.

In some embodiments, the chemical reagents add a functional group to the paired nucleobase. In some embodiments, the functional group is added to guanine. In some embodiments, the functional group is added to an oxygen atom of guanine. In some embodiments, the functional group is selected from hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, the functional group is optionally substituted C₁-C₃ alkyl-C-carboxy or optionally substituted C₇-C₁₂ aralkyl. In some embodiments, the functional group is —CH₂C(O)OR³ and R³ is methyl, ethyl, or t-butyl. In some embodiments, the functional group is —CH₂C(O)OEt. In some embodiments, the functional group is optionally substituted benzyl. In some embodiments, the functional group is benzyl.

Some embodiments include forming the copy polynucleotide strand by the use of one or more sulfur-containing nucleotides. In some embodiments, the sulfur-containing nucleotide is selected from thio-dATP, thio-dCTP, thio-dGTP, thio-dTTP, and combinations of any of the foregoing. In some embodiments, the sulfur-containing nucleotide is thio-dGTP. In some embodiments, the sulfur-containing nucleotide is 6-thioguanine deoxynucleotide triphosphate. The sequential addition of one or more sulfur-containing nucleotides to the copy nucleotide strand forms a sulfur-containing copy nucleotide strand that is complementary to the target polynucleotide strand. The sulfur-containing nucleotide comprises a sulfur-containing paired nucleobase. In some embodiments, the sulfur-containing paired nucleobase is selected from thioadenine, thiocytosine, thioguanine, thiothymine, and combinations of any of the foregoing. In some embodiments, the sulfur-containing paired nucleobase is thiogaunine. In some embodiments, the sulfur-containing paired nucleobase is 6-thioguanine. In some embodiments, the sulfur-containing paired nucleobase forms a base pair with the modified nucleobase of the target polynucleotide strand.

Some embodiments include converting the sulfur-containing paired nucleobase accomplished with chemical reagents. In some embodiments, the chemical reagents include oxidizing agents, nucleophiles, or combinations of any of the foregoing. In some embodiments, the chemical reagents include one or more oxidizing agents. In some embodiments, the oxidizing agent is an inorganic salt. In some embodiments, the oxidizing agent comprises chromate, hypervalent halide, hypohalide, peroxide, peroxy acid, peroxy salt, or combinations of any of the foregoing. In some embodiments, the oxidizing agent comprises NaIO₄. In some embodiments, the chemical reagents include one or more nucleophiles. In some embodiments, the nucleophile is selected from a nitrogen-containing nucleophile, an oxygen-containing nucleophile, a sulfur-containing nucleophile, and combinations of any of the foregoing. In some embodiments, the nucleophile has the formula R⁴B¹, wherein B¹ is NH₂, OH, or SH and R⁴ is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, C₅-C₁₂ heteroaralkyl, and combinations of any of the foregoing. In some embodiments, R⁴ is selected from C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl. In some embodiments, the nucleophile is selected from alanine, phenol, thiophenol, benzyl amine, benzyl alcohol, and benzyl mercaptan. In some embodiments, the nucleophile is benzyl amine. In some embodiments, the nucleophile is benzyl alcohol. In some embodiments, the nucleophile is benzyl mercaptan.

In some embodiments, the chemical reagents add a functional group to the sulfur-containing paired nucleobase. In some embodiments, the functional group is added to a sulfur-containing guanine. In some embodiments, the functional group is added to a 6-sulfonylguanine. In some embodiments, the functional group is added to a carbon atom of guanine. In some embodiments, the functional group has the formula R⁴B², wherein B² is NH, O, or S and R⁴ is selected from C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, C₅-C₁₂ heteroaralkyl, and combinations of any of the foregoing. In some embodiments, R⁴ is C₆-C₁₂ aryl or C₇-C₁₂ aralkyl. In some embodiments, R⁴ is selected from NHPh, OPh, SPh, NHCH₂Ph, OCH₂Ph, and SCH₂Ph. In some embodiments, the functional group is NHCH₂Ph, OCH₂Ph, or SCH₂Ph. In some embodiments, the functional group is OCH₂Ph.

In some embodiments, the sulfur-containing paired nucleobase is treated with the chemical reagents in a stepwise fashion. In some embodiments, the sulfur-containing paired nucleobase is first treated with the oxidizing agent to produce an intermediate sulfur-containing paired nucleobase that is contacted with the nucleophile in a second step. For example, the sulfur-containing paired nucleobase 6-thioguanine can be oxidized to 6-sulfonylguanine. In a second step the 6-sulfonylguanine can be contacted with a benzyl alcohol to initiate a nucleophilic aromatic substitution reaction. The product of the nucleophilic aromatic substitution is an orthogonal nucleobase comprising 6-O-benzylguanine.

Some embodiments include a polymerase that is configured to incorporate the signal nucleotide into the signal nucleotide strand. In some embodiments, the signal nucleotide strand complementary to the copy polynucleotide strand. In some embodiments, the polymerase is a DNA polymerase or an RNA polymerase. In some embodiments, the polymerase is a naturally occurring polymerase, a mutant polymerase, or a rationally engineered polymerase. In some embodiments, the polymerase comprises an A-family DNA polymerase, a B-family DNA polymerase, a Y-family DNA polymerase, and combinations of any of the foregoing. In some embodiments, the polymerase is a mutant DNA polymerase. In some embodiments, the polymerase is selected from Dpo4, Therminator, DeepVent® (exo−), KOD, KlenTaq, KTqM747K, and combinations of any of the foregoing, as described in Wyss et al., “Specific Incorporation of an Artificial Nucleotide Opposite a Mutagenic DNA Adduct by a DNA Polymerase”, 2015, J. Am. Chem. Soc., 137, 30-33. In some embodiments, the polymerase is Dpo4. In some embodiments, the polymerase is Therminator. In some embodiments, the polymerase is DeepVent® (exo-). In some embodiments, the second polymerase is KOD. In some embodiments, the polymerase is KlenTaq. In some embodiments, the polymerase is KTqM747K.

### Method of Detecting: Alternate 3^{rd} Base Pair

In some embodiments, the methods include converting the modified nucleobase into a linked signal nucleobase. It will be appreciated that the methods that follow are related to the previously described methods illustrated in FIGS. 1-11. The description of the methods that follow can be understood in view of the methods previously described elsewhere herein. For example, the step of converting the modified nucleobase in the presently described method occurs after the step of providing the target polynucleotide strand and occurs instead of the steps of forming a copy polynucleotide strand comprising a paired nucleobase and removing the modified nucleobase.

The term “linked signal nucleobase,” as used herein is a signal nucleobase that is converted, or otherwise formed, from a modified nucleobase that was not removed from a target nucleotide strand. In some embodiments, the methods include converting the plurality of modified nucleobases into a plurality of linked signal nucleobases. In some embodiments, the linked signal nucleobase comprises a derivative of the modified nucleobase. In some embodiments, the linked signal nucleobase comprises a derivative of 5-hydroxymethylcytosine, e.g., a bicyclic derivative of 5-hydroxymethylcytosine containing a six membered oxazine ring. In some embodiments, the linked signal nucleobase has the structure:

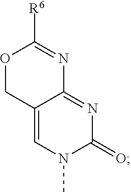

wherein “ [symbol] ” is a bond to the signal polynucleotide strand. In some embodiments, R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, R⁶ is C₁-C₆ alkyl. In some embodiments, R⁶ is methyl, ethyl, or propyl.

In some embodiments, the converting is a two-step process that includes an enzymatic process and a chemical process. The two-step process includes the enzymatic process occurring before or after the chemical process. In some embodiments, the methods include contacting the modified nucleobase with an enzyme. The enzyme is configured to convert the modified nucleobase selectively in the presence of other nucleobases. In some embodiments, the enzyme may be a dioxygenase, non-limiting examples of which include a ten-eleven translocation (TET) methylcytosine dioxygenase. Contacting with the enzyme forms a derivatized modified nucleobase. In some embodiments, the methods include contacting the modified nucleobase with a TET methylcytosine dioxygenase.

In some embodiments, the derivatized modified nucleobase is 5-hydroxymethylcytosine. In some embodiments, the modified nucleobase is 5-methylcytosine and the derivatized modified nucleobase is 5-hydroxymethylcytosine.

The methods include contacting the derivatized modified nucleobase with a chemical reagent to form the linked signal nucleobase. In some embodiments, the chemical reagent is a chemical reagent configured for alkylation, acetylation, cycloaddition, elimination, isomerization, oxidation, reduction, substitution, or any combination of the foregoing. In some embodiments, the chemical reagent is an acidic reagent, non-limiting examples of which include an acid chloride. In some embodiments, the chemical reagent is acetyl chloride. In some embodiments, the methods include contacting the derivatized modified nucleobase with acetyl chloride to form a six membered oxazine ring of the linked signal nucleobase.

In some embodiments, the modified nucleobase is 5-methylcytosine and the methods include contacting with a TET methylcytosine dioxygenase then contacting with acetyl chloride. In some embodiments, the linked signal nucleobase has the structure:

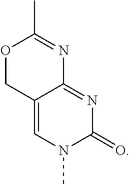

In some embodiments, the methods include incorporating at least one orthogonal nucleotide into the copy polynucleotide strand. In some embodiments, the copy polynucleotide strand is a growing copy polynucleotide strand. In some embodiments, the methods include incorporating a plurality of orthogonal nucleotides into the growing copy polynucleotide strand. The copy polynucleotide strand is complementary to at least a portion of the target polynucleotide strand that comprises the at least one linked signal nucleobase.

The orthogonal nucleotide includes a linked orthogonal nucleobase. In some embodiments, the linked orthogonal nucleobase comprises a purine or a derivative thereof. The linked orthogonal nucleobase is configured to achieve Watson-Crick base pairing with the linked signal nucleobase. In some embodiments, the linked orthogonal nucleobase has a structure selected from:

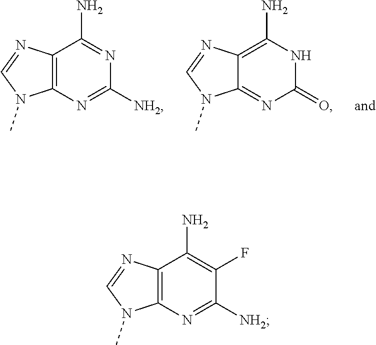

wherein “ [symbol] ” is a bond to the copy polynucleotide strand. In some embodiments, the orthogonal nucleotide includes a detectable label.

In some embodiments, the methods include incorporating a signal nucleotide into a growing signal polynucleotide strand. In some embodiments, the signal polynucleotide strand is a growing signal polynucleotide strand. In some embodiments, the methods include incorporating a plurality of signal nucleotides into the growing signal polynucleotide strand. The signal polynucleotide strand is complementary to at least a portion of the copy polynucleotide strand that comprises the at least one orthogonal nucleotide. The signal nucleotide includes the linked signal nucleobase, as described elsewhere herein.

The linked signal nucleobase achieves Watson-Crick base pairing with the linked orthogonal nucleobase and thereby creates a third DNA base pair. In some embodiments, the linked signal nucleobase achieves Watson-Crick base pairing with the orthogonal nucleobase and thereby creates a third DNA base pair. In some embodiments, the linked signal nucleobase does not achieve Watson-Crick base pairing with the orthogonal nucleobase. The linked signal nucleobase does not achieve Watson-Crick base pairing with a natural nucleobase.

In some embodiments, the linked orthogonal nucleobase achieves Watson-Crick base pairing with the signal nucleobase and thereby creates a third DNA base pair. In some embodiments, the linked orthogonal nucleobase does not achieve Watson-Crick base pairing with the signal nucleobase. The linked orthogonal nucleobase does not achieve Watson-Crick base pairing with a natural nucleobase.

The signal nucleotide comprising the linked signal nucleobase includes a detectable label, as described elsewhere herein. The identity of a newly incorporated signal nucleotide comprising the linked signal nucleobase is determined with the detectable label and allows for detection of the modified nucleobase in the target polynucleotide strand. The identity of the linked signal nucleobase corresponds to the identity modified nucleobase because the linked signal nucleobase and the modified nucleobase occupy the same position in the target polynucleotide strand. In other words, detecting the modified nucleobase in the target polynucleotide strand is accomplished with the detectable label of the newly incorporated signal nucleotide comprising the linked signal nucleobase.

### Method of Forming a Six-Nucleobase Polynucleotide

Some embodiments of the present disclosure relate to methods of forming a six-base polynucleotide. It will be appreciated that the methods of forming a six-base polynucleotide that follow are related to the previously described methods illustrated in FIGS. 1-11. The description of the methods that follow can be understood in view of the methods previously described elsewhere herein. In some embodiments, the six-base polynucleotide comprises a polynucleotide or an oligonucleotide. In some embodiments, the six-base polynucleotide comprises a signal polynucleotide strand and copy polynucleotide strand. In some embodiments, the signal polynucleotide strand comprises a DNA strand or an RNA stand.

In certain embodiments, the signal polynucleotide strand of the six-base polynucleotide includes a plurality of signal nucleobases. In some embodiments, the signal nucleobase comprises a structure selected from the group consisting of:

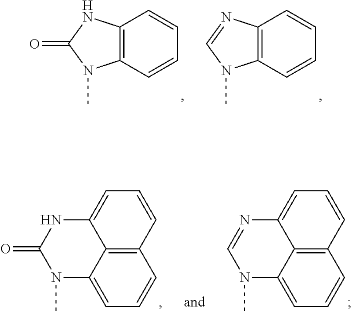

wherein “ [symbol] ” is a bond to the signal polynucleotide strand. The signal nucleobase does not achieve Watson-Crick base pairing with a linked orthogonal nucleobase or a natural nucleobase.

In certain embodiments, the copy polynucleotide strand of the six-base polynucleotide includes a plurality of orthogonal nucleobases. In some embodiments, the orthogonal nucleobase has the structure selected from:

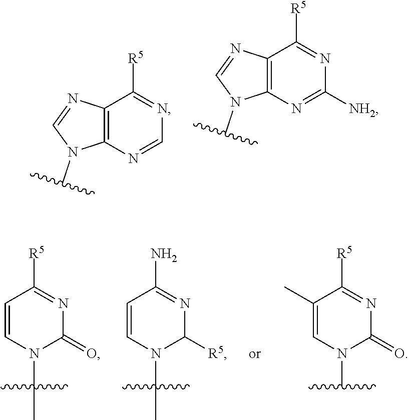

In some embodiments, R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, R³ is selected from optionally substituted C₁-C₃ alkyl-C-carboxy or optionally substituted C₇-C₁₂ aralkyl. In some embodiments, R⁵ is CH₂C(O)OR³ and R³ is methyl, ethyl, or t-butyl. In some embodiments, R⁵ is CH₂C(O)OEt. In some embodiments, R⁵ is NHPh, OPh, SPh, NHCH₂Ph, OCH₂Ph, or SCH₂Ph. In some embodiments, R⁵ is OCH₂Ph. In some embodiments, R⁵ is phenyl or benzyl. In some embodiments, the orthogonal nucleobase is O-benzylguanine. In some embodiments, an orthogonal nucleobase comprises at least one functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, C₅-C₁₂ heteroaralkyl and any combination of the foregoing. The orthogonal nucleobase may achieve Watson-Crick base pairing with the signal nucleobase. The functional group on the orthogonal nucleobase allows the orthogonal nucleobase to achieve Watson-Crick base pairing selectively with the structural features of the signal nucleobase and form a third DNA base pair. The third DNA base pair creates the six-nucleobase polynucleotide. The functional group on the orthogonal nucleobase prevents Watson-Crick base pairing with a natural nucleobase. In some embodiments, the orthogonal nucleobase does not achieve Watson-Crick base pairing with a linked signal nucleobase or a natural nucleobase.

The method of forming the six-base polynucleotide includes providing a target polynucleotide strand that includes the plurality of modified nucleobases. In some embodiments, the modified nucleobase may be selected from any of the modified nucleobases as described elsewhere herein. In some embodiments, the modified nucleobase is 5-methylcytosine.

In certain embodiments, the method of forming the six-base polynucleotide includes forming the copy polynucleotide strand that includes the plurality of paired nucleobases. In some embodiments, the paired nucleobase may be selected from any of the paired nucleobases as described elsewhere herein. In some embodiments, the paired nucleobase is guanine. The method includes removing the plurality of modified nucleobases. In some embodiments, removing is accomplished any of the glycosylases as described elsewhere herein. In some embodiments, the glycosylase removes the plurality of modified nucleobases to form a gapped polynucleotide strand as described elsewhere herein. The method includes converting the plurality of paired nucleobases into the plurality of orthogonal nucleobases. In some embodiments, converting is accomplished with any of the chemical reagents as described elsewhere herein. In some embodiments, the chemical reagents include a diazo compound, a metal catalyst, and a reducing agent. In some embodiments, the chemical reagents add a plurality of functional groups to the plurality of paired nucleobases. In some embodiments, the plurality of functional groups is added to a plurality of oxygen atoms of guanine. In some embodiments, the functional group is benzyl.

In certain embodiments, the method of forming the six-base polynucleotide includes using sulfur-containing nucleotides to form the copy polynucleotide strand that includes a plurality of sulfur-containing paired nucleobases, as described elsewhere herein. In some embodiments, the sulfur-containing nucleotide is 6-thioguanine deoxynucleotide triphosphate. In some embodiments, the sulfur-containing paired nucleobase is 6-thioguanine. The method includes removing the plurality of modified nucleobases. In some embodiments, removing is accomplished any of the glycosylases as described elsewhere herein. In some embodiments, the glycosylase removes the plurality of modified nucleobases to form a gapped polynucleotide strand as described elsewhere herein. The method includes converting a plurality of sulfur-containing paired nucleobases with any of the chemical reagents as described elsewhere herein. In some embodiments, the chemical reagents include one or more oxidizing agents and one or more nucleophiles. In some embodiments, the chemical reagents convert the plurality of sulfur-containing paired nucleobases into a plurality of orthogonal nucleobases comprising 6-O-benzylguanine.

The method of forming the six-base polynucleotide includes incorporating the plurality of signal nucleobases into the signal polynucleotide strand. Some embodiments include a polymerase that is configured to incorporate the signal nucleotide into the signal nucleotide strand as described elsewhere herein. In some embodiments, the polymerase is selected from Dpo4, Therminator, DeepVent® (exo−), KOD, KlenTaq, KTqM747K, and any combination of the foregoing.

In other embodiments, the signal polynucleotide strand of the six-base polynucleotide includes a plurality of linked signal nucleobases. In some embodiments, the linked signal nucleobase has the structure:

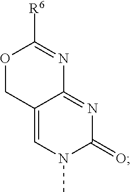

wherein “ [symbol] ” is a bond to the signal polynucleotide strand. In some embodiments, R⁶ is selected [symbol] from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₂ cycloalkyl, 3-10 membered heterocyclyl, C₁-C₁₂ aryl, C₇-C₁₂ aralkyl, 5-10 membered heteroaryl, C₅-C₁₂ heteroaralkyl, and optionally substituted derivatives of any of the foregoing. In some embodiments, R⁶ is C₁-C₆ alkyl. In some embodiments, R⁶ is methyl, ethyl, or propyl.

In other embodiments, the copy polynucleotide strand of the six-base polynucleotide includes a plurality of linked orthogonal nucleobases. In some embodiments, the linked orthogonal nucleobase comprises a purine or a derivative thereof. The linked orthogonal nucleobase is configured to achieve Watson-Crick base pairing with the linked signal nucleobase. In some embodiments, the linked orthogonal nucleobase has a structure selected from:

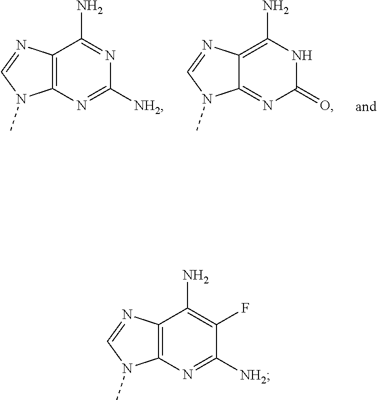

wherein “ [symbol] ” is a bond to the copy polynucleotide strand. In some embodiments, the orthogonal nucleotide includes a detectable label.

In other embodiments, the method of forming the six-base polynucleotide includes converting the plurality of modified nucleobases into the plurality of linked signal nucleobases. The converting is a two-step process that includes an enzymatic process and a chemical process, as previously described herein. In some embodiments, the methods include contacting the plurality of modified nucleobases with a TET methylcytosine dioxygenase then contacting with acetyl chloride. In some embodiments, each of the plurality of signal nucleobases has the structure:

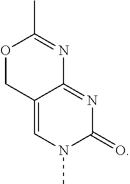

In other embodiments, the method of forming the six-base polynucleotide includes incorporating a plurality of linked orthogonal nucleotides into the copy polynucleotide strand. The linked orthogonal nucleotide comprises the linked orthogonal nucleobase having a structure selected from.

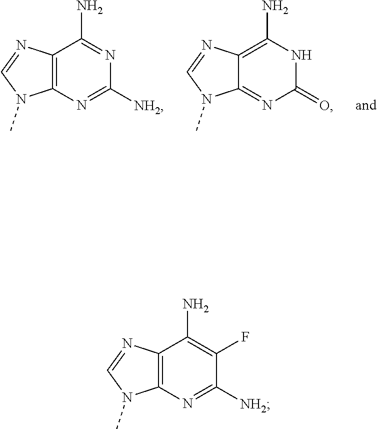

wherein “ [symbol] ” is a bond to the copy polynucleotide strand. In some embodiments, the orthogonal [symbol] nucleotide includes a detectable label.

In other embodiments, the method of forming the six-base polynucleotide includes incorporating the plurality of signal nucleotides into the signal polynucleotide strand. Some embodiments include a polymerase that is configured to incorporate the plurality of signal nucleotide into the signal nucleotide strand as described elsewhere herein. In some embodiments, the polymerase is selected from Dpo4, Therminator, DeepVent® (exo−), KOD, KlenTaq, KTqM747K, and any combination of the foregoing.

### Six-Nucleobase Polynucleotides

Some embodiments of the present disclosure relate to a six-nucleobase polynucleotide. In some embodiments, the six-nucleobase polynucleotide includes a signal polynucleotide strand and a copy polynucleotide strand. In some embodiments, the signal polynucleotide strand includes a plurality of signal nucleobases. In some embodiments, the copy polynucleotide strand includes a plurality of orthogonal nucleobases. In some embodiments, a signal nucleobase comprises a structure selected from the group consisting of:

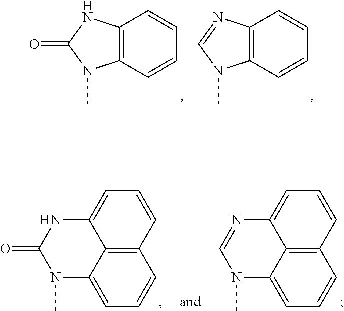

wherein “ [symbol] ” is a bond to the signal polynucleotide strand. In some embodiments, an orthogonal nucleobase includes a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl. In some embodiments, the orthogonal nucleobase achieves Watson-Crick base pairing with the signal nucleobase.

In some embodiments, the signal nucleobase comprises the structure:

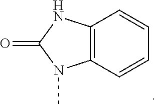

Tn some embodiments, the signal nucleobase does not achieve Watson-Crick base pairing with a natural nucleobase.

In some embodiments, the orthogonal nucleobase has the structure selected from:

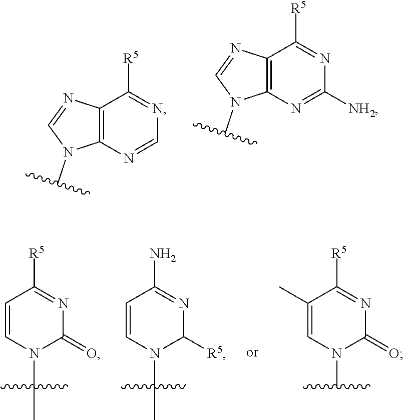

and
wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl. In some embodiments, the orthogonal nucleobase is O-benzylguanine. In some embodiments, the orthogonal nucleobase does not achieve Watson-Crick base pairing with a natural nucleobase.

In other embodiments, the signal polynucleotide strand of the six-nucleobase polynucleotide includes a plurality of linked signal nucleobases. In some embodiments, a linked signal nucleobase has the structure:

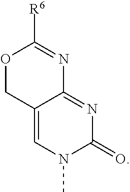

In some embodiments, R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing. In some embodiments, R⁶ is C₁-C₆ alkyl. In some embodiments, R⁶ is methyl, ethyl, or propyl. In some embodiments, “ [symbol] ” is a bond to the signal polynucleotide strand.

In some embodiments, the linked signal nucleobase comprises the structure:

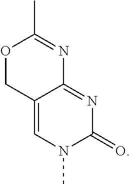

In some embodiments, the linked signal nucleobase does not achieve Watson-Crick base pairing with a natural nucleobase.

Tn other embodiments, the copy polynucleotide strand of the six-nucleobase polynucleotide includes a plurality of linked orthogonal nucleobases. In some embodiments, a linked orthogonal nucleobase has a structure selected from the group consisting of:

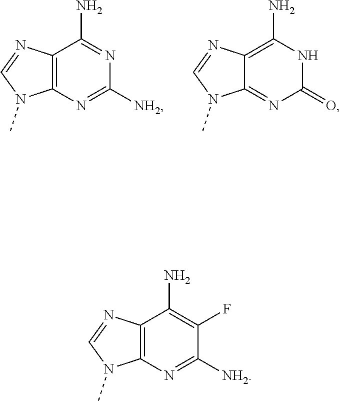

Tn some embodiments, “ [symbol] ” is a bond to the copy polynucleotide strand.

The linked orthogonal nucleobase achieves Watson-Crick base pairing with the linked signal nucleobase. In some embodiments, the linked orthogonal nucleobase does not achieve Watson-Crick base pairing with a natural nucleobase.

### Six-Nucleobase Nucleotides and Nucleosides

Some further embodiments of the present disclosure relate to six-nucleobase nucleotides and six-nucleobase nucleosides. The terms “six-nucleobase nucleotide” and “six-nucleobase nucleoside” refer to a nucleotide or a nucleoside, respectively, comprising one or more orthogonal nucleobases and one or more signal nucleobases, as described elsewhere herein. The six-nucleobase nucleotide or six-nucleobase nucleoside may be covalently attached to a detectable label (for example, a fluorophore), optionally via a linker. The linker may be cleavable or non-cleavable. In some embodiments, the six-nucleobase nucleotide or six-nucleobase nucleoside further comprises a 3′ hydroxy blocking group.

In some embodiments, the 3′ hydroxy blocking group and the cleavable linker (and the attached label) may be removed under the same or substantially same chemical reaction conditions, for example, the blocking group and the detectable label may be removed in a single chemical reaction. In other embodiments, the blocking group and the detectable labeled are removed in two separate steps.

In some embodiments, the six-nucleobase nucleotides or six-nucleobase nucleosides described herein comprises 2′ deoxyribose. In some further aspects, the 2′ deoxyribose contains one, two or three phosphate groups at the 5′ position of the sugar ring. In some further aspect, the nucleotides described herein are nucleotide triphosphate.

Compatibility with Linearization

In order to maximize the throughput of nucleic acid sequencing reactions it is advantageous to be able to sequence multiple template molecules in parallel. Parallel processing of multiple templates can be achieved with the use of nucleic acid array technology. These arrays typically consist of a high-density matrix of polynucleotides immobilized onto a solid support material.

PCT Publication Nos. WO 98/44151 and WO 00/18957 both describe methods of nucleic acid amplification which allow amplification products to be immobilized on a solid support in order to form arrays comprised of clusters or “colonies” formed from a plurality of identical immobilized polynucleotide strands and a plurality of identical immobilized complementary strands. Arrays of this type are referred to herein as “clustered arrays.” The nucleic acid molecules present in DNA colonies on the clustered arrays prepared according to these methods can provide templates for sequencing reactions, for example as described in WO 98/44152. The products of solid-phase amplification reactions such as those described in WO 98/44151 and WO 00/18957 are so-called “bridged” structures formed by annealing of pairs of immobilized polynucleotide strands and immobilized complementary strands, both strands being attached to the solid support at the 5′ end. In order to provide more suitable templates for nucleic acid sequencing, it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure is referred to as “linearization.” There are various ways for linearization, including but not limited to enzymatic cleavage, photo-chemical cleavage, or chemical cleavage. Non-limiting examples of linearization methods are disclosed in PCT Publication No. WO 2007/010251, U.S. Patent Publication No. 2009/0088327, U.S. Patent Publication No. 2009/0118128, and U.S. Appl. 62/671,816, which are incorporated by reference in their entireties.

In some embodiments, the six-nucleobase nucleotides and six-nucleobase nucleosides comprising the orthogonal nucleobases and signal nucleobases described herein are compatible with the linearization processes.

Unless indicated otherwise, the reference to six-nucleobase nucleotides is also intended to be applicable to six-nucleobase nucleosides.

### Labeled Nucleotides

According to an aspect of the disclosure, nucleotides or nucleosides, including the six-nucleobase nucleotides and six-nucleobase nucleosides described herein, also comprise a detectable label and such nucleotide is called a labeled nucleotide. The label (e.g., a fluorescent dye) can be conjugated via an optional linker by a variety of means including hydrophobic attraction, ionic attraction, and covalent attachment. In some aspects, the dyes are conjugated to the substrate by covalent attachment. More particularly, the covalent attachment is by means of a linker group. In some instances, such labeled nucleotides are also referred to as “modified nucleotides.”

Labeled nucleosides and nucleotides are useful for labeling polynucleotides formed by enzymatic synthesis, such as, by way of non-limiting example, in PCR amplification, isothermal amplification, solid phase amplification, polynucleotide sequencing (e.g., solid phase sequencing), nick translation reactions and the like.

In some embodiments, the dye may be covalently attached to oligonucleotides or nucleotides via the nucleotide base. For example, the labeled nucleotide or oligonucleotide may have the label attached to the C5 position of a pyrimidine base or the C7 position of a 7-deaza purine base through a linker moiety.

Unless indicated otherwise, the reference to six-nucleobase nucleotides is also intended to be applicable to six-nucleobase nucleosides. The present application will also be further described with reference to DNA, although the description will also be applicable to RNA, PNA, and other nucleic acids, unless otherwise indicated.

Nucleotides or nucleosides, including the six-nucleobase nucleotides and six-nucleobase nucleosides described herein, may be labeled at sites on the sugar or nucleobase. Although the nucleobase is usually referred to as a purine or pyrimidine, the skilled person will appreciate that derivatives and analogues are available which do not alter the capability of the nucleotide or nucleoside to undergo Watson-Crick base pairing. “Derivative” or “analogue” means a compound or molecule whose core structure is the same as, or closely resembles that of a parent compound, but which has a chemical or physical modification, such as, for example, a different or additional side group, which allows the derivative nucleotide or nucleoside to be linked to another molecule. For example, the nucleobase may be a deazapurine. In particular embodiments, the derivatives should be capable of undergoing Watson-Crick base pairing. “Derivative” and “analogue” also include, for example, a synthetic nucleotide or nucleoside derivative having modified base moieties and/or modified sugar moieties. Such derivatives and analogues are discussed in, for example, Scheit, Nucleotide analogs (John Wiley & Son, 1980) and Uhlman et al., Chemical Reviews 90:543-584, 1990. Nucleotide analogues can also comprise modified phosphodiester linkages including phosphorothioate, phosphorodithioate, alkyl-phosphonate, phosphoranilidate, phosphoramidate linkages and the like.

In particular embodiments the labeled nucleoside or nucleotide may be enzymatically incorporable and enzymatically extendable. Accordingly, a linker moiety may be of sufficient length to connect the nucleotide to the compound such that the compound does not significantly interfere with the overall binding and recognition of the nucleotide by a nucleic acid replication enzyme. Thus, the linker can also comprise a spacer unit. The spacer distances, for example, the nucleotide base from a cleavage site or label.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

### Methods of Sequencing

Labeled nucleotides or nucleosides according to the present disclosure may be used in any method of analysis such as method that include detection of a fluorescent label attached to a nucleotide or nucleoside, including the six-nucleobase nucleotides and six-nucleobase nucleosides described herein, whether on its own or incorporated into or associated with a larger molecular structure or conjugate. In this context the term “incorporated into a polynucleotide” can mean that the 5′ phosphate is joined in phosphodiester linkage to the 3′-OH group of a second (modified or unmodified) nucleotide, which may itself form part of a longer polynucleotide chain. The 3′ end of a nucleotide set forth herein may or may not be joined in phosphodiester linkage to the 5′ phosphate of a further (modified or unmodified) nucleotide. Thus, in one non-limiting embodiment, the disclosure provides a method of detecting a nucleotide (e.g., six-nucleobase nucleotide), incorporated into a polynucleotide which comprises: (a) incorporating at least one six-nucleobase nucleotide of the disclosure into a polynucleotide and (b) detecting the six-nucleobase nucleotide(s) incorporated into the polynucleotide by detecting the fluorescent signal from the dye compound attached to said six-nucleobase nucleotide(s).

This method can include: a synthetic step (a) in which one or more six-nucleobase nucleotides according to the disclosure are incorporated into a polynucleotide and a detection step (b) in which one or more six-nucleobase nucleotide(s) incorporated into the polynucleotide are detected by detecting or quantitatively measuring their fluorescence.

Some embodiments of the present application are directed to methods of sequencing including: (a) incorporating at least one labeled six-nucleobase nucleotide as described herein into a polynucleotide; and (b) detecting the labeled six-nucleobase nucleotide(s) incorporated into the polynucleotide by detecting the fluorescent signal from the new fluorescent dye attached to said six-nucleobase nucleotide(s).

Some embodiments of the present disclosure relate to a method for determining the sequence of a target single-stranded polynucleotide, comprising:
(a) incorporating a six-nucleobase nucleotide comprising a 3′-OH blocking group and a detectable label as described herein into a copy polynucleotide strand complementary to at least a portion of the target polynucleotide strand;
(b) detecting the identity of the six-nucleobase nucleotide incorporated into the copy polynucleotide strand; and
(c) chemically removing the label and the 3′-OH blocking group from the six-nucleobase nucleotide incorporated into the copy polynucleotide strand.

In some embodiments, the sequencing method further comprises (d) washing the chemically removed label and the 3′ blocking group away from the copy polynucleotide strand. In some such embodiments, the 3′ blocking group and the detectable label are removed prior to introducing the next complementary nucleotide. In some further embodiments, the 3′ blocking group and the detectable label are removed in a single step of chemical reaction. In some embodiment, the washing step (d) also remove unincorporated nucleotides. In some further embodiments, a palladium scavenger is also used in the washing step after chemical cleavage of the label and the 3′ blocking group.

In some embodiments, steps (a) to (d) are repeated until a sequence of the portion of the template polynucleotide strand is determined. In some such embodiments, steps (a) to (d) are repeated at least 50 times, at least 75 times, at least 100 times, at least 150 times, at least 200 times, at least 250 times, or at least 300 times.

Tn any embodiments of the methods described herein, the labeled six-nucleobase nucleotide is a six-nucleobase nucleotide triphosphate. In any embodiments of the method described herein, the target polynucleotide strand is attached to a solid support, such as a flow cell.

Tn one embodiment, at least one six-nucleobase nucleotide is incorporated into a six-nucleobase polynucleotide in the synthetic step by the action of a polymerase. In some such embodiments, the polymerase may be DNA polymerase Pol 812 or Pol 1901. In other such embodiments, the polymerase is a mutant DNA polymerase selected from Dpo4, Therminator, DeepVent® (exo−), KOD, KlenTaq, KTqM747K, and combinations of any of the foregoing. However, other methods of joining six-nucleobase nucleotides to six-nucleobase polynucleotides, such as, for example, chemical oligonucleotide synthesis or ligation of labeled oligonucleotides to unlabeled oligonucleotides, can be used. Therefore, the term “incorporating,” when used in reference to a six-nucleobase nucleotide and six-nucleobase polynucleotide, can encompass polynucleotide synthesis by chemical methods as well as enzymatic methods.

In a specific embodiment, a synthetic step is carried out and may optionally comprise incubating a template polynucleotide strand with a reaction mixture comprising labeled six-nucleobase nucleotides of the disclosure. A polymerase can also be provided under conditions which permit formation of a phosphodiester linkage between a free 3′-OH group on a polynucleotide strand annealed to the template polynucleotide strand and a 5′ phosphate group on the six-nucleobase nucleotide. Thus, a synthetic step can include formation of a polynucleotide strand as directed by complementary base-pairing of six-nucleobase nucleotides to a template strand.

In all embodiments of the methods, the detection step may be carried out while the polynucleotide strand into which the labeled six-nucleobase nucleotides are incorporated is annealed to a template strand, or after a denaturation step in which the two strands are separated. Further steps, for example chemical or enzymatic reaction steps or purification steps, may be included between the synthetic step and the detection step. In particular, the target strand incorporating the labeled six-nucleobase nucleotide(s) may be isolated or purified and then processed further or used in a subsequent analysis. By way of example, target polynucleotides labeled with six-nucleobase nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including nucleotides as described herein, to form an extended target strand complementary to the template strand in the presence of a suitable polymerase enzyme. In other embodiments, the synthetic step may itself form part of an amplification reaction producing a labeled double stranded amplification product comprised of annealed complementary strands derived from copying of the target and template polynucleotide strands. Other exemplary synthetic steps include nick translation, strand displacement polymerization, random primed DNA labeling, etc. A particularly useful polymerase enzyme for a synthetic step is one that is capable of catalyzing the incorporation of six-nucleobase nucleotides as set forth herein. A variety of naturally occurring or modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the six-nucleobase nucleotides according to the disclosure include those described in WO 2005/024010 or WO 06/120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity, and the like.

In specific non-limiting embodiments, the disclosure encompasses methods of nucleic acid sequencing, re-sequencing, whole genome sequencing, single nucleotide polymorphism scoring, any other application involving the detection of the labeled six-nucleobase nucleotide or six-nucleobase nucleoside set forth herein when incorporated into a polynucleotide. Any of a variety of other applications benefitting the use of polynucleotides labeled with the six-nucleobase nucleotides comprising fluorescent dyes can use labeled six-nucleobase nucleotides or six-nucleobase nucleosides with dyes set forth herein.

In a particular embodiment, the disclosure provides use of labeled six-nucleobase nucleotides according to the disclosure in a polynucleotide sequencing-by-synthesis (SBS) reaction. Sequencing-by-synthesis generally involves sequential addition of one or more six-nucleobase nucleotides or oligonucleotides to a growing polynucleotide chain in the 5′ to 3′ direction using a polymerase or ligase in order to form an extended polynucleotide chain complementary to the template nucleic acid to be sequenced. The identity of the base present in one or more of the added six-nucleobase nucleotide(s) can be determined in a detection or “imaging” step. The identity of the added base may be determined after each six-nucleobase nucleotide incorporation step. The sequence of the template may then be inferred using conventional Watson-Crick base-pairing rules. The use of the labeled six-nucleobase nucleotides set forth herein for determination of the identity of a single base (e.g., modified nucleobase), may be useful, for example, in the scoring of single nucleotide polymorphisms, and such single base extension reactions are within the scope of this disclosure.

In an embodiment of the present disclosure, the sequence of a template polynucleotide is determined by detecting the incorporation of one or more 3′ blocked six-nucleobase nucleotides described herein into a nascent strand complementary to the template polynucleotide to be sequenced through the detection of fluorescent label(s) attached to the incorporated six-nucleobase nucleotide(s). Sequencing of the template polynucleotide can be primed with a suitable primer (or prepared as a hairpin construct which will contain the primer as part of the hairpin), and the nascent chain is extended in a stepwise manner by addition of six-nucleobase nucleotides to the 3′ end of the primer in a polymerase-catalyzed reaction.

In particular embodiments, each of the different natural and six-nucleobase nucleotide triphosphates may be labeled with a unique fluorophore and also comprises a blocking group at the 3′ position to prevent uncontrolled polymerization. Alternatively, one of the natural and six-nucleobase nucleotides may be unlabeled (dark). The polymerase enzyme incorporates a natural or six-nucleobase nucleotide into the nascent chain complementary to the template polynucleotide, and the blocking group prevents further incorporation of nucleotides. Any unincorporated nucleotides can be washed away and the fluorescent signal from each incorporated nucleotide can be “read” optically by suitable means, such as a charge-coupled device using laser excitation and suitable emission filters. The 3′-blocking group and fluorescent dye compounds can then be removed (deprotected) simultaneously or sequentially to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, different natural A, G, C, and T and six-nucleobase 3′-blocked nucleotides into a growing strand complementary to the immobilized polynucleotide, in the presence of DNA polymerase. The polymerase incorporates a base complementary to the target polynucleotide but is prevented from further addition by the 3′-blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3-OH group that serves as a primer or initiation point for the addition of further nucleotides in the sequencing reaction. The region of the template to be sequenced will overhang this free 3′-OH group on the complementary strand. The overhanging region of the template to be sequenced may be single stranded but can be double-stranded, provided that a “nick is present” on the strand complementary to the template strand to be sequenced to provide a free 3′-OH group for initiation of the sequencing reaction. In such embodiments, sequencing may proceed by strand displacement. In certain embodiments, a primer bearing the free 3′-OH group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO 01/57248 and WO 2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′-OH group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

Tn certain embodiments, the nucleic acid template to be sequenced may be attached to a solid support via any suitable linkage method known in the art, for example via covalent attachment. In certain embodiments template polynucleotides may be attached directly to a solid support (e.g., a silica-based support). However, in other embodiments of the disclosure the surface of the solid support may be modified in some way so as to allow either direct covalent attachment of template polynucleotides, or to immobilize the template polynucleotides through a hydrogel or polyelectrolyte multilayer, which may itself be non-covalently attached to the solid support.

### Embodiments and Alternatives of Sequencing-by-Synthesis

Some embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina, Inc.), and is also described in WO 91/06678 and WO 07/123,744, each of which is incorporated herein by reference. The availability of fluorescently labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed, and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

Some embodiments can utilize detection of six different nucleotides using fewer than six different labels. For example, SBS can be performed utilizing methods and systems described in the incorporated materials of U.S. Pub. No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification, or physical modification) that causes apparent signal to appear or disappear compared to the signal detected for the other member of the pair. As a second example, five of six different nucleotide types can be detected under particular conditions while a sixth nucleotide type lacks a label that is detectable under those conditions, or is minimally detected under those conditions (e.g., minimal detection due to background fluorescence, etc.). Incorporation of the first five nucleotide types into a nucleic acid can be determined based on presence of their respective signals and incorporation of the sixth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Pub. No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”, Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some other embodiments of sequencing method involve the use the six-nucleobase nucleotides described herein in nanoball sequencing technique, such as those described in U.S. Pat. No. 9,222,132, the disclosure of which is incorporated by reference. Through the process of rolling circle amplification (RCA), a large number of discrete DNA nanoballs may be generated. The nanoball mixture is then distributed onto a patterned slide surface containing features that allow a single nanoball to associate with each location. In DNA nanoball generation, DNA is fragmented and ligated to the first of four adapter sequences. The template is amplified, circularized, and cleaved with a type II endonuclease. A second set of adapters is added, followed by amplification, circularization, and cleavage. This process is repeated for the remaining two adapters. The final product is a circular template with four adapters, each separated by a template sequence. Library molecules undergo a rolling circle amplification step, generating a large mass of concatemers called DNA nanoballs, which are then deposited on a flow cell. Goodwin et al., “Coming of age: ten years of next-generation sequencing technologies,” Nat Rev Genet. 2016; 17(6):333-51.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414, both of which are incorporated herein by reference, or nucleotide incorporations can be detected with zero-mode waveguides as described, for example, in U.S. Pat. No. 7,315,019, which is incorporated herein by reference, and using fluorescent nucleotide analogs and engineered polymerases as described, for example, in U.S. Pat. No. 7,405,281 and U.S. Pub. No. 2008/0108082, both of which are incorporated herein by reference. The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in U.S. Pub. Nos. 2009/0026082; 2009/0127589; 2010/0137143; and 2010/0282617, all of which are incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for detection of target nucleic acids. Exemplary flow cells are described, for example, in U.S. Pub. No. 2010/0111768 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

Arrays in which polynucleotides have been directly attached to silica-based supports are those for example disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported template polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178, and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732(2005), each of which is incorporated herein by reference. Sequencing of arrays of such beads using nucleotides set forth herein is within the scope of the disclosure.

Templates that are to be sequenced may form part of an “array” on a solid support, in which case the array may take any convenient form. Thus, the method of the disclosure is applicable to all types of high-density arrays, including single-molecule arrays, clustered arrays, and bead arrays. Labeled nucleotides of the present disclosure may be used for sequencing templates on essentially any type of array, including but not limited to those formed by immobilization of nucleic acid molecules on a solid support.

However, labeled nucleotides of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using the nucleotides labeled with dye compounds of the disclosure.

The labeled nucleotides of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the nucleotides of the disclosure is in sequencing-by-synthesis reactions, the utility of the nucleotides is not limited to such methods. In fact, the nucleotides may be used advantageously in any sequencing methodology which requires detection of fluorescent labels attached to nucleotides incorporated into a polynucleotide.

Some embodiments relate to the following enumerated alternatives:

1. A method of detecting a modified nucleobase in a target polynucleotide strand, comprising: providing a target polynucleotide strand comprising the modified nucleobase; forming a copy polynucleotide strand comprising a paired nucleobase; removing the modified nucleobase; converting the paired nucleobase into an orthogonal nucleobase; and incorporating a signal nucleotide into a signal polynucleotide strand, wherein the signal nucleotide comprises a signal nucleobase and a detectable label.

2. The method of alternative 1, wherein the signal nucleobase comprises the structure:

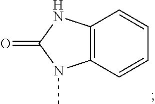

wherein “ [symbol] ” is a bond to the signal polynucleotide strand.

3. The method of any one of alternatives 1-2, wherein the signal nucleobase does not achieve Watson-Crick base pairing with a natural nucleobase.

4. The method of any one of alternatives 1-3, wherein the orthogonal nucleobase comprises:

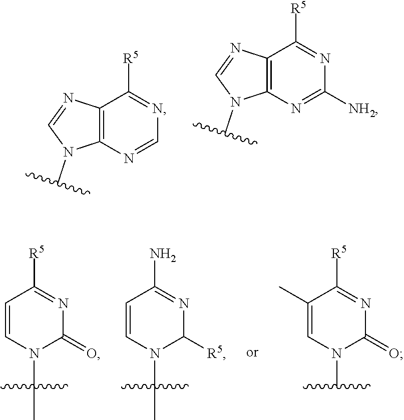

and wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

5. The method of any one of alternatives 1-4, wherein the orthogonal nucleobase is O-benzylguanine.

6. The method of any one of alternatives 1-5, wherein the orthogonal nucleobase does not achieve Watson-Crick base pairing with a natural nucleobase.

7. The method of any one of alternatives 1-6, wherein the orthogonal nucleobase achieves Watson-Crick base pairing with the signal nucleobase.

8. The method of any one of alternatives 1-7, wherein the modified nucleobase is selected from the group consisting of a modified adenine, a modified cytosine, a modified guanine, and a modified thymine, and a modified uracil.

9. The method of one of alternatives 1-8, wherein the removing is accomplished by a glycosylase selected from the group consisting of ROS1 DNA glycosylase, DME DNA glycosylase, DML2 DNA glycosylase, and DML3 DNA glycosylase.

10. The method of any one of alternatives 1-9, wherein converting the paired nucleobase is accomplished with chemical reagents, the chemical reagents comprising a diazo compound having the structure N₂CWZ, wherein W is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, or an optionally substituted derivative of any of the foregoing; Z is selected from C(O)NR¹R², C(O)OR¹, C(O)SR¹, C(S)OR¹, and C(S)SR¹; and R¹ and R² are independently selected from C₁-C₁₂ alkyl, C₂-C₁₂ alkenyl, C₂-C₁₂ alkynyl, C₁-C₁₂ alkoxy, C₁-C₁₂ heteroalkyl, cyano, halo, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, C₁-C₁₂ thioalkyl, C₁-C₁₂ sulfonyl, or an optionally substituted derivative of any of the foregoing, wherein R¹ and R² together optionally are 3-10 membered heterocyclyl or 5-10 membered heteroaryl.

11. The method of alternative 10, wherein the chemical reagents add a functional group to the paired nucleobase, the functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

12. The method of any one of alternatives 1-11, wherein the copy polynucleotide strand is a sulfur-containing copy nucleotide strand and forming the sulfur-containing copy polynucleotide strand is accomplished with 6-thioguanine deoxynucleotide triphosphate.

13. The method of alternative 12, wherein the paired nucleobase is a sulfur-containing paired nucleobase and converting the sulfur-containing paired nucleobase is accomplished with chemical reagents, the chemical reagents comprising one or more oxidizing agents and a nucleophile having the formula R⁴B¹, wherein B¹ is NH₂, OH, or SH and R⁴ is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl.

14. The method of alternative 13, wherein the chemical reagents add a functional group to the sulfur-containing paired nucleobase, the functional group having the formula R⁴B², wherein B² is NH, O, or S and R⁴ is selected from C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl.

15. The method of any one of alternatives 1-14, wherein incorporating the signal nucleobase into the signal polynucleotide strand is accomplished by a polymerase selected from the group consisting of Dpo4, Therminator, DeepVent® (exo−), KOD, KlenTaq, and KTqM747K.

16. A method of detecting a modified nucleobase in a target polynucleotide strand, the method comprising: providing a target polynucleotide strand comprising the modified nucleobase; converting the modified nucleobase into a linked signal nucleobase; incorporating an orthogonal nucleotide into a copy polynucleotide strand, the orthogonal nucleotide comprising a linked orthogonal nucleobase; and incorporating a signal nucleotide into a signal polynucleotide strand, the signal nucleotide comprising the linked signal nucleobase and a detectable label.

17. The method of alternative 16, wherein the linked signal nucleobase has the structure:

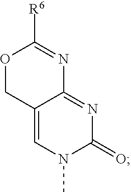

wherein R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing, and “ [symbol] ” is a bond to the signal polynucleotide strand.

18. The method of any one of alternatives 16-17, wherein the liked orthogonal nucleobase has the structure:

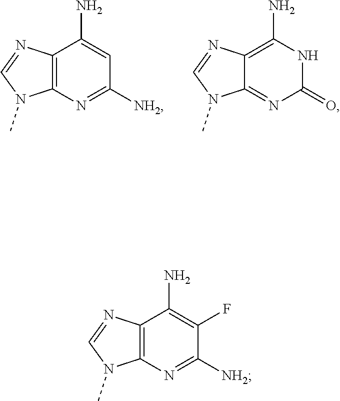

wherein “ [symbol] ” is a bond to the copy polynucleotide strand.

19. A method of forming a six-nucleobase polynucleotide comprising a signal polynucleotide strand and a copy polynucleotide strand, the signal polynucleotide strand comprising a plurality of signal nucleobases and the copy polynucleotide strand comprising a plurality of orthogonal nucleobases, wherein a signal nucleobase comprises a structure selected from the group consisting of:

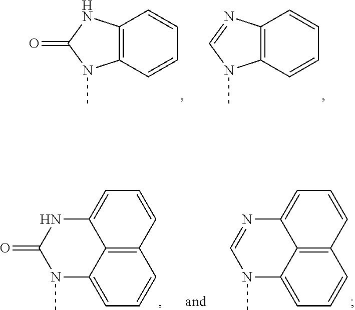

wherein “ [symbol] ” is a bond to the signal polynucleotide strand; and an orthogonal nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl and the orthogonal nucleobase achieves Watson-Crick base pairing with the signal nucleobase, the method comprising: providing a target polynucleotide strand comprising the plurality of modified nucleobases; forming the copy polynucleotide strand; removing the plurality of modified nucleobases; converting the plurality of paired nucleobases into the plurality of orthogonal nucleobases; and incorporating the plurality of signal nucleobases into the signal polynucleotide strand.

20. A method of forming a six-nucleobase polynucleotide comprising a signal polynucleotide strand and a copy polynucleotide strand, the signal polynucleotide strand comprising a plurality of linked signal nucleobases and the copy polynucleotide strand comprising a plurality of linked orthogonal nucleobases, wherein a linked signal nucleobase has the structure:

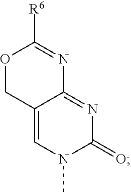

wherein R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing, “ [symbol] ” is a bond to the signal polynucleotide strand, and a linked orthogonal nucleobase has a structure selected from the group consisting of:

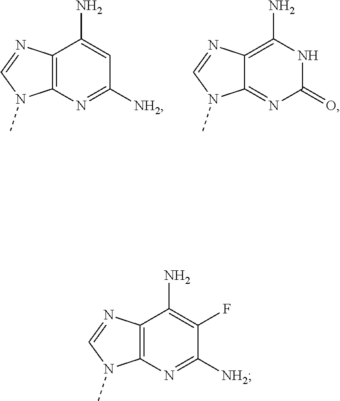

wherein “ [symbol] ” is a bond to the copy polynucleotide strand, the method comprising: providing a target polynucleotide strand comprising the plurality of modified nucleobases; converting the plurality of modified nucleobases into the plurality of linked signal nucleobases; incorporating a plurality of orthogonal nucleotides into the copy polynucleotide strand, wherein an orthogonal nucleotide comprises the linked orthogonal nucleobase; and incorporating a plurality of signal nucleotides into the signal polynucleotide strand, wherein a signal nucleotide comprises the linked signal nucleobase and a detectable label.

21. A six-nucleobase polynucleotide comprising a signal polynucleotide strand and a copy polynucleotide strand, wherein the signal polynucleotide strand comprises a plurality of signal nucleobases and the copy polynucleotide strand comprises a plurality of orthogonal nucleobases, wherein a signal nucleobase comprises a structure selected from the group consisting of:

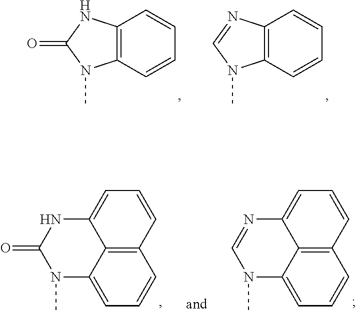

wherein “ [symbol] ” is a bond to the signal polynucleotide strand, wherein an orthogonal nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl, and wherein the orthogonal nucleobase achieves Watson-Crick base pairing with the signal nucleobase.

22. The six-nucleobase polynucleotide of alternative 21, wherein the signal nucleobase comprises the structure:

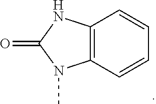

23. The six-nucleobase polynucleotide of any one of alternatives 21-22, wherein the signal nucleobase does not achieve Watson-Crick base pairing with a natural nucleobase.

24. The six-nucleobase polynucleotide of any one of alternatives 22-23, wherein the orthogonal nucleobase has the structure selected from:

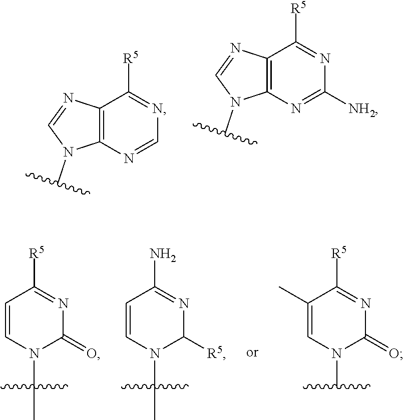

and wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

25. The six-nucleobase polynucleotide of any one of alternatives 21-24, wherein the orthogonal nucleobase is O-benzylguanine.

26. The six-nucleobase polynucleotide of any one of alternatives 21-25, wherein the orthogonal nucleobase does not achieve Watson-Crick base pairing with a natural nucleobase.

27. A six-nucleobase polynucleotide comprising a signal polynucleotide strand and a copy polynucleotide strand, wherein the signal polynucleotide strand comprises a plurality of linked signal nucleobases and the copy polynucleotide strand comprises a plurality of linked orthogonal nucleobases, wherein a linked signal nucleobase has the structure:

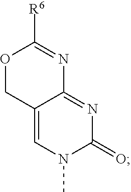

wherein R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing, “ [symbol] ” is a bond to the signal polynucleotide strand, a linked orthogonal nucleobase has a structure selected from the group consisting of:

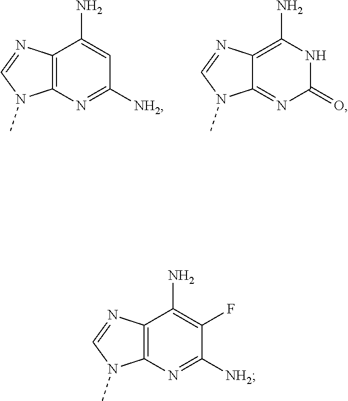

wherein “ [symbol] ” is a bond to the copy polynucleotide strand, and wherein the linked orthogonal nucleobase achieves Watson-Crick base pairing with the linked signal nucleobase.

28. The six-nucleobase polynucleotide of alternative 27, wherein the linked signal nucleobase comprises the structure:

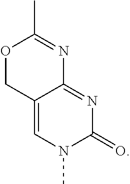

29. The six-nucleobase polynucleotide of any one of alternatives 27-28, wherein the linked signal nucleobase does not achieve Watson-Crick base pairing with a natural nucleobase.

30. The six-nucleobase polynucleotide of any one of alternatives 27-29, wherein the linked orthogonal nucleobase does not achieve Watson-Crick base pairing with a natural nucleobase.

### EXAMPLES

Additional embodiments are disclosed in further detail in the following examples, which are not in any way intended to limit the scope of the claims.

### Example 1—Six-Base Amplification and Sequencing

The following example demonstrates methods for six-base amplification and sequencing to detect the presence of methylated nucleotides in a polynucleotide.

A bead-linked transposome (BLT) was provided. Methylated forms of double-stranded DNA (dsDNA) fragments were provided and mixed with the BLT to bind the dsDNA to the BLT for transposition, as shown in FIG. 3.

The transposase and non-transfer Tsn strand were removed. A Hybe Y-adapter, with GFL to attached to 3′ ends were inserted as an anchor extension primer. The primer was bound to the 3′ end of the Y-adapter. Extension from primer was achieved using a DNA polymerase, as shown in FIG. 4.

The sample was treated with a 5-methyl cytosine (5mC) specific glycosylase (such as ROS1), which cleaved the 5mC from the DNA duplex, leaving a 1-bp gap, as shown in FIG. 5.

The DNA duplex was mixed with chemical reagents, which react with the guanine, specifically at gapped positions to alter base pairing from cytosine to an orthogonal base, as shown in FIG. 6.

The primer bound to the anchor strand and an engineered DNA polymerase was used to incorporate an orthogonal partner base opposite the modified guanine. Amplification was performed either linearly or exponentially, as shown in FIG. 10.

Six-base DNA polymerases were used to generate clusters on a flow cell, followed by six-base SBS with two additional FFNs, as shown in FIG. 11.

All publications, patents, and patent applications mentioned in this specification are herein incorporated by reference to the same extent as if each individual publication, patent, or patent application was specifically and individually indicated to be incorporated by reference.

While preferred embodiments described herein have been shown and described herein, it will be obvious to those skilled in the art that such embodiments are provided by way of example only. Numerous variations, changes, and substitutions will now occur to those skilled in the art without departing from the description. It should be understood that various alternatives to the embodiments described herein may be employed in practicing the embodiments. It is intended that the following claims define the scope of embodiments provided herein and that methods and structures within the scope of these claims and their equivalents be covered thereby.

What is claimed is:

1. A method of detecting a modified nucleobase in a target polynucleotide strand, comprising:
providing a target polynucleotide strand comprising the modified nucleobase;
forming a copy polynucleotide strand comprising a paired nucleobase;
removing the modified nucleobase;
converting the paired nucleobase into an orthogonal nucleobase; and
incorporating a signal nucleotide into a signal polynucleotide strand, wherein the signal nucleotide comprises a signal nucleobase and a detectable label.

2. The method of claim 1, wherein the signal nucleobase comprises the structure: 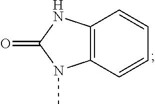
wherein “ [symbol] ” is a bond to the signal polynucleotide strand.

3. The method of claim 1, wherein the signal nucleobase does not achieve Watson-Crick base pairing with a natural nucleobase.

4. The method of claim 1, wherein the orthogonal nucleobase comprises: 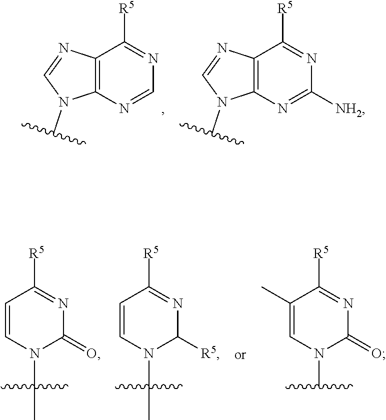
wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

5. The method of claim 1, wherein the orthogonal nucleobase is O-benzylguanine.

6. The method of claim 1, wherein the orthogonal nucleobase does not achieve Watson-Crick base pairing with a natural nucleobase.

7. The method of claim 1, wherein the orthogonal nucleobase achieves Watson-Crick base pairing with the signal nucleobase.

8. The method of claim 1, wherein the modified nucleobase comprises a modified adenine, a modified cytosine, a modified guanine, and a modified thymine, or a modified uracil.

9. The method of claim 1, wherein the removing is accomplished by a glycosylase comprising ROS1 DNA glycosylase, DME DNA glycosylase, DML2 DNA glycosylase, or DML3 DNA glycosylase.

10. The method of claim 1, wherein converting the paired nucleobase is accomplished with chemical reagents, the chemical reagents comprising a diazo compound having the structure N₂CWZ,
wherein W is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, or an optionally substituted derivative of any of the foregoing;
Z is selected from C(O)NR¹R², C(O)OR¹, C(O)SR¹, C(S)OR¹, and C(S)SR¹; and
R¹ and R² are independently selected from C₁-C₁₂ alkyl, C₂-C₁₂ alkenyl, C₂-C₁₂ alkynyl, C₁-C₁₂ alkoxy, C₁-C₁₂ heteroalkyl, cyano, halo, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, C₁-C₁₂ thioalkyl, C₁-C₁₂ sulfonyl, or an optionally substituted derivative of any of the foregoing,
wherein R¹ and R² together optionally are 3-10 membered heterocyclyl or 5-10 membered heteroaryl.

11. The method of claim 10, wherein the chemical reagents add a functional group to the paired nucleobase, the functional group comprising hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, or C₅-C₁₂ heteroaralkyl.

12. The method of claim 1, wherein the copy polynucleotide strand is a sulfur-containing copy nucleotide strand and forming the sulfur-containing copy polynucleotide strand is accomplished with 6-thioguanine deoxynucleotide triphosphate.

13. The method of claim 12, wherein the paired nucleobase is a sulfur-containing paired nucleobase and converting the sulfur-containing paired nucleobase is accomplished with chemical reagents, the chemical reagents comprising one or more oxidizing agents and a nucleophile having the formula R⁴B¹, wherein B¹ is NH₂, OH, or SH and R⁴ is selected from H, C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl.

14. The method of claim 13, wherein the chemical reagents add a functional group to the sulfur-containing paired nucleobase, the functional group having the formula R⁴B², wherein B² is NH, O, or S and R⁴ is selected from C₁-C₁₂ alkyl, C₂-C₁₂ alkenyl, C₂-C₁₂ alkynyl, C₄-C₁₂ carbocyclyl, C₄-C₁₂ cycloalkyl, C₆-C₁₂ aryl, C₇-C₁₂ aralkyl, 5-10 membered heteroaryl, and C₅-C₁₂ heteroaralkyl.

15. The method of claim 1, wherein incorporating the signal nucleobase into the signal polynucleotide strand is accomplished by a polymerase selected from the group consisting of Dpo4, Therminator, DeepVent® (exo−), KOD, KlenTaq, and KTqM747K.

16. A method of detecting a modified nucleobase in a target polynucleotide strand, the method comprising:
providing a target polynucleotide strand comprising the modified nucleobase;
converting the modified nucleobase into a linked signal nucleobase;
incorporating an orthogonal nucleotide into a copy polynucleotide strand, the orthogonal nucleotide comprising a linked orthogonal nucleobase; and
incorporating a signal nucleotide into a signal polynucleotide strand, the signal nucleotide comprising the linked signal nucleobase and a detectable label.

17. The method of claim 16, wherein the linked signal nucleobase has the structure: 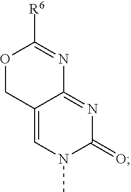
wherein
R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing, and
“ [symbol] ” is a bond to the signal polynucleotide strand.

18. The method of claim 16, wherein the liked orthogonal nucleobase has the structure: 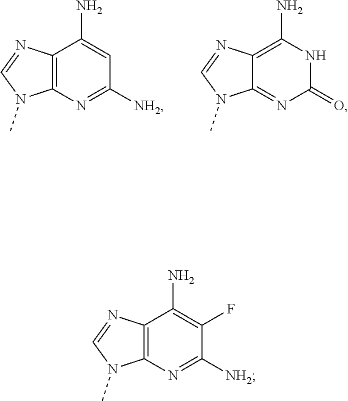
wherein “ [symbol] ” is a bond to the copy polynucleotide strand.

19. A method of forming a six-nucleobase polynucleotide comprising a signal polynucleotide strand and a copy polynucleotide strand, the signal polynucleotide strand comprising a plurality of signal nucleobases and the copy polynucleotide strand comprising a plurality of orthogonal nucleobases,
wherein a signal nucleobase comprises a structure selected from the group consisting of: 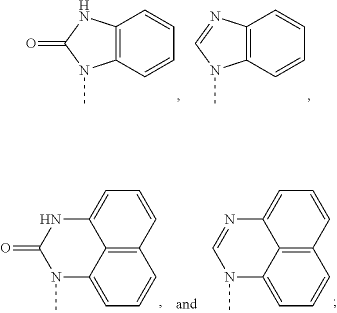
wherein “ [symbol] ” is a bond to the signal polynucleotide strand; and
an orthogonal nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl and the orthogonal nucleobase achieves Watson-Crick base pairing with the signal nucleobase,
the method comprising:
providing a target polynucleotide strand comprising the plurality of modified nucleobases;
forming the copy polynucleotide strand;
removing the plurality of modified nucleobases;
converting the plurality of paired nucleobases into the plurality of orthogonal nucleobases; and
incorporating the plurality of signal nucleobases into the signal polynucleotide strand.

20. A method of forming a six-nucleobase polynucleotide comprising a signal polynucleotide strand and a copy polynucleotide strand, the signal polynucleotide strand comprising a plurality of linked signal nucleobases and the copy polynucleotide strand comprising a plurality of linked orthogonal nucleobases,
wherein a linked signal nucleobase has the structure: 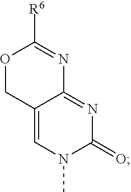
wherein
R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing,
“ [symbol] ” is a bond to the signal polynucleotide strand, and
a linked orthogonal nucleobase has a structure selected from the group consisting of: 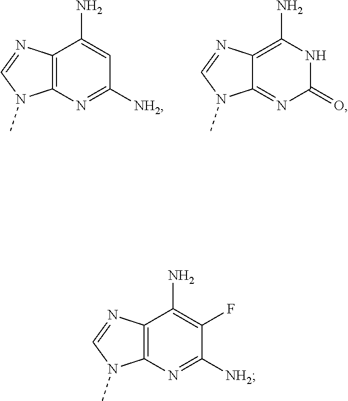
wherein “ [symbol] ” is a bond to the copy polynucleotide strand,
the method compising:
providing a target polynucleotide strand comprising the plurality of modified nucleobases;
converting the plurality of modified nucleobases into the plurality of linked signal nucleobases;
incorporating a plurality of orthogonal nucleotides into the copy polynucleotide strand, wherein an orthogonal nucleotide comprises the linked orthogonal nucleobase; and
incorporating a plurality of signal nucleotides into the signal polynucleotide strand, wherein a signal nucleotide comprises the linked signal nucleobase and a detectable label.

21. A six-nucleobase polynucleotide comprising a signal polynucleotide strand and a copy polynucleotide strand, wherein the signal polynucleotide strand comprises a plurality of signal nucleobases and the copy polynucleotide strand comprises a plurality of orthogonal nucleobases,
wherein a signal nucleobase comprises a structure selected from the group consisting of: 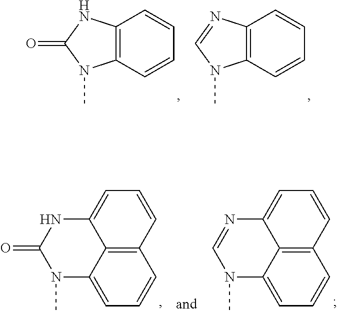
wherein “ [symbol] ” is a bond to the signal polynucleotide strand,
wherein an orthogonal nucleobase comprises a functional group selected from the group consisting of hydroxy, cyano, halo, C₁-C₆ alkyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ arylalkyl, C₇-C₁₂ arylalkoxy, 5-10 membered heteroaryl, and C₅-C₁₂ heteroaralkyl, and
wherein the orthogonal nucleobase achieves Watson-Crick base pairing with the signal nucleobase.

22. The six-nucleobase polynucleotide of claim 21, wherein the signal nucleobase comprises the structure:

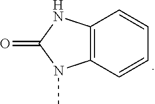

23. The six-nucleobase polynucleotide of claim 21, wherein the signal nucleobase does not achieve Watson-Crick base pairing with a natural nucleobase.

24. The six-nucleobase polynucleotide of claim 22, wherein the orthogonal nucleobase has the structure selected from: 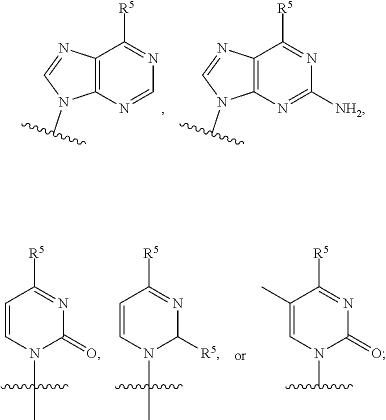
wherein R⁵ is selected from the group consisting of hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, and C₅-C₁₂ heteroaralkyl.

25. The six-nucleobase polynucleotide of claim 21, wherein the orthogonal nucleobase is O-benzylguanine.

26. The six-nucleobase polynucleotide of claim 21, wherein the orthogonal nucleobase does not achieve Watson-Crick base pairing with a natural nucleobase.

27. A six-nucleobase polynucleotide comprising a signal polynucleotide strand and a copy polynucleotide strand, wherein the signal polynucleotide strand comprises a plurality of linked signal nucleobases and the copy polynucleotide strand comprises a plurality of linked orthogonal nucleobases,
wherein a linked signal nucleobase has the structure: 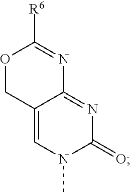
wherein
R⁶ is selected from the group consisting of hydrogen, hydroxy, cyano, halo, C₁-C₆ alkyl, C₂-C₆ alkenyl, C₂-C₆ alkynyl, C₁-C₃ alkyl-C-carboxy, C₁-C₆ alkoxy, C₄-C₁₂ carbocyclyl, C₄-C₁₂ cycloalkyl, 3-10 membered heterocyclyl, C₆-C₁₂ aryl, C₇-C₁₂ aralkyl, 5-10 membered heteroaryl, C₅-C₁₂ heteroaralkyl, and optionally substituted derivatives of any of the foregoing,
“ [symbol] ” is a bond to the signal polynucleotide strand,
a linked orthogonal nucleobase has a structure selected from the group consisting of: 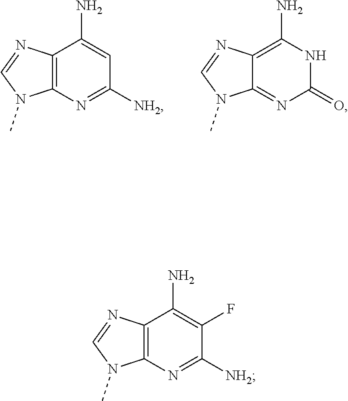
wherein “ [symbol] ” is a bond to the copy polynucleotide strand, and
wherein the linked orthogonal nucleobase achieves Watson-Crick base pairing with the linked signal nucleobase.

28. The six-nucleobase polynucleotide of claim 27, wherein the linked signal nucleobase comprises the structure:

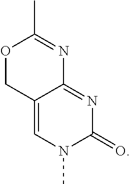

29. The six-nucleobase polynucleotide of claim 27, wherein the linked signal nucleobase does not achieve Watson-Crick base pairing with a natural nucleobase.

30. The six-nucleobase polynucleotide of claim 27, wherein the linked orthogonal nucleobase does not achieve Watson-Crick base pairing with a natural nucleobase.
